(19) United States
(12) Patent Application Publication
(10) Pub. No.: US 2024/0350654 A1
(43) Pub. Date: Oct. 24, 2024

(54) NANOCARRIERS FOR TARGETED DELIVERY OF THERAPEUTICS

(71) Applicant: Northwestern University, Evanston, IL (US)

(72) Inventors: Clayton Hunter Rische, Evanston, IL (US); Bruce Scott Bochner, Evanston, IL (US); Evan Alexander Scott, Evanston, IL (US)

(21) Appl. No.: 18/639,549

(22) Filed: Apr. 18, 2024

Related U.S. Application Data
(60) Provisional application No. 63/496,874, filed on Apr. 18, 2023.

(51) Int. Cl. A61K 47/68; A61K 9/51; A61K 47/69; A61P 37/06

(52) U.S. Cl. CPC A61K 47/6849; A61K 9/5192; A61K 47/6935; A61P 37/06

(57) ABSTRACT

Provided herein are therapeutic PPSU nanoparticles for targeting specific cell types and delivering therapeutic compounds. Therapeutic PPSU nanoparticles include nanoparticles comprising midostaurin and anti-siglec antibodies for targeting mast cells. Also provided herein are methods for preparing the therapeutic PPU nanoparticles and using them to treat malignant mast cell disorders.

### CROSS-REFERENCE TO RELATED APPLICATIONS

This application claims the benefit of and priority to U.S. Provisional Application No. 63/496,874 filed on Apr. 18, 2023, the content of which is incorporated by reference in its entirety.

### STATEMENT REGARDING FEDERALLY SPONSORED RESEARCH

This invention was made with government support under A1159586 awarded by the National Institutes of Health. The government has certain rights in the invention.

### REFERENCE TO A SEQUENCE LISTING

The contents of the electronic sequence listing (702581.02501.xml; Size: 113,700 bytes; and Date of Creation: Apr. 16, 2024) is herein incorporated by reference in its entirety.

### BACKGROUND

Mast cells are uniquely long-lived granulocytes that are omnipresent throughout the body and fundamental to both the innate and adaptive immune response. Despite their many beneficial immunological roles, dysregulated mast cell responses contribute to a wide range of pathologies that include allergy, anaphylaxis, and cancer. A major contemporary therapeutic question remains as to whether it is possible to selectively target mast cells and their functions directly in all tissues. Healthy mast cells do not usually proliferate but do exist in tissue-specific subclasses. Pathologically, this implies that subpopulations of mast cells may respond differently depending on the therapy being applied and the disease context. This highlights the critical need for targeted therapies that can penetrate tissues to reach specific mast cell populations to address disease without disrupting immune homeostasis.

Given their contributions to disease, mast cells are logical therapeutic targets for a wide range of pathologies including cancer. For example, mast cells can become malignant in a disease known as systemic mastocytosis. This is caused by aberrations in the KIT gene, resulting in gain-of-function mutations that allow for their clonal proliferation independent of stimulation with the KIT receptor ligand, stem cell factor (SCF). By far the most common version of the mutation occurs in exon 17 of the gene and is known as D816V. Mastocytosis has been classified in several forms, some of which are life-threatening, while isolated mastocytomas tend to be self-limited. Additionally, a role for mast cells in non-mast cell solid tumors also seems likely, so targeting mast cells may influence other types of cancer. These classifications, in order of relative severity, are cutaneous mastocytosis, indolent systemic mastocytosis, systemic smoldering mastocytosis, aggressive systemic mastocytosis, mast cell leukemia, and mast cell sarcoma. Another classification, known as systemic mastocytosis with associated hematologic neoplasm, is as severe and aggressive as other advanced forms of the disease, but displays a wider range in severity due to the variable nature of the associated neoplasms. Mutations in the KIT gene are the most reliable characteristics of malignant mast cells, however histological studies of cell morphology and growth combined with detection of aberrantly expressed surface markers such as CD25, and perhaps to a lesser extent CD2, are part of the criteria needed to define mastocytosis. As a result, bone marrow biopsies are commonly needed, but the invasiveness of this analysis requires reasonable evidence that mastocytosis is present. Despite this knowledge of mast cell function, identification markers, and roles in a wide range of immunopathology, few therapies that directly target these cells have been successfully developed.

Mast cells have proven to be difficult targets for direct and selective drug treatment. This is due to a combination of factors: 1) they have only a few unique cell surface and intracellular targets, and fewer are readily receptive to safe and selective modulation, 2) mast cells are long-lived, senescent or slowly dividing tissue-resident cells present throughout the body, 3) many aspects of their responsiveness and mediators are interrelated with products from other cells (e.g., IgE, SCF) and have effects on other immune cells as well as non-immune cells like the vasculature and neurons. As a result, potential anti-mast cell therapeutics have side effects due to low specificity (e.g., KIT is expressed by cells other than mast cells) or indirectly target mast cells by antagonizing their mediators, with very few, if any, true “mast cell stabilizing” or specific mast cell depleting drugs, despite the panoply of receptors on these cells. Therefore, improved mast cell-targeting therapeutics that cause minimal side effects are needed.

### SUMMARY

In a first aspect, provided herein is a composition for depleting mast cells, the composition comprising: a PPSU nanoparticle leaded with midostaurin and coated with an anti-siglec 6 antibody; and a pharmaceutically acceptable carrier.

In embodiments, the anti-siglec 6 antibody is selected from: antibody clone 767329; and an antibody comprising: a CDRH1 comprising a sequence selected from SEQ ID NOs: 89, 17, 83, 5, 11, 77, 71, 168, 53, 29, 23, 35, 41, 47, 59, 65, 135, 141, 147; a CDRH2 comprising a sequence selected from SEQ ID NOs: 90, 18, 84, 6, 12, 78, 72, 169, 54, 30, 24, 36, 42, 48, 60, 66, 136, 142, 148; a CDRH3 comprising a sequence selected from SEQ ID NOs: 91, 19, 85, 7, 13, 79, 73, 7, 55, 31, 25, 37, 43, 49, 61, 67, 137, 143, 149; a CDRL1 comprising a sequence selected from SEQ ID NOs: 92, 20, 86, 8, 14, 80, 74, 8, 56, 32, 26, 38, 44, 50, 62, 68, 138, 144, 150; a CDRL2 comprising a sequence selected from SEQ ID NOs: 93, 21, 87, 9, 15, 81, 75, 9, 57, 33, 27, 39, 45, 51, 63, 69, 139, 145, 151; and a CDRL3 comprising a sequence selected from SEQ ID NOs: 94, 22, 88, 10, 16, 82, 76, 10, 58, 34, 28, 40, 46, 52, 64, 70, 140, 146, 152.

In embodiments, the anti-siglec 6 antibody comprises: (a) the CDRH1 sequence comprising SEQ ID NO: 89; the CDRH2 sequence comprising SEQ ID NO: 90; the CDRH3 comprising SEQ ID NO: 91; the CDRL1 sequence comprising SEQ ID NO: 92; the CDRL2 sequence comprising SEQ ID NO: 93; and the CDRL3 sequence comprising SEQ ID NO: 94; (b) the CDRH1 sequence comprising SEQ ID NO: 17; the CDRH2 sequence comprising SEQ ID NO: 18; the CDRH3 comprising SEQ ID NO: 19; the CDRL1 sequence comprising SEQ ID NO: 20; the CDRL2 sequence comprising SEQ ID NO: 21; and the CDRL3 sequence comprising SEQ ID NO: 22; (c) the CDRH1 sequence comprising SEQ ID NO: 83; the CDRH2 sequence comprising SEQ ID NO: 84; the CDRH3 comprising SEQ ID NO: 85; the CDRL1 sequence comprising SEQ ID NO: 86; the CDRL2 sequence comprising SEQ ID NO: 87; and the CDRL3 sequence comprising SEQ ID NO: 88; (d) the CDRH1 sequence comprising SEQ ID NO: 5; the CDRH2 sequence comprising SEQ ID NO: 6; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10; (e) the CDRH1 sequence comprising SEQ ID NO: 11; the CDRH2 sequence comprising SEQ ID NO: 12; the CDRH3 comprising SEQ ID NO: 13; the CDRL1 sequence comprising SEQ ID NO: 14; the CDRL2 sequence comprising SEQ ID NO: 15; and the CDRL3 sequence comprising SEQ ID NO: 16; (f) the CDRH1 sequence comprising SEQ ID NO: 77; the CDRH2 sequence comprising SEQ ID NO: 78; the CDRH3 comprising SEQ ID NO: 79; the CDRL1 sequence comprising SEQ ID NO: 80; the CDRL2 sequence comprising SEQ ID NO: 81; and the CDRL3 sequence comprising SEQ ID NO: 82; (g) the CDRH1 sequence comprising SEQ ID NO: 71; the CDRH2 sequence comprising SEQ ID NO: 72; the CDRH3 comprising SEQ ID NO: 73; the CDRL1 sequence comprising SEQ ID NO: 74; the CDRL2 sequence comprising SEQ ID NO: 75; and the CDRL3 sequence comprising SEQ ID NO: 76; (h) the CDRH1 sequence comprising SEQ ID NO: 168; the CDRH2 sequence comprising SEQ ID NO: 169; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10; (i) the CDRH1 sequence comprising SEQ ID NO: 53; the CDRH2 sequence comprising SEQ ID NO: 54; the CDRH3 comprising SEQ ID NO: 55; the CDRL1 sequence comprising SEQ ID NO: 56; the CDRL2 sequence comprising SEQ ID NO: 57; and the CDRL3 sequence comprising SEQ ID NO: 58; (j) the CDRH1 sequence comprising SEQ ID NO: 29; the CDRH2 sequence comprising SEQ ID NO: 30; the CDRH3 comprising SEQ ID NO: 31; the CDRL1 sequence comprising SEQ ID NO: 32; the CDRL2 sequence comprising SEQ ID NO: 33; and the CDRL3 sequence comprising SEQ ID NO: 34; (k) the CDRH1 sequence comprising SEQ ID NO: 23; the CDRH2 sequence comprising SEQ ID NO: 24; the CDRH3 comprising SEQ ID NO: 25; the CDRL1 sequence comprising SEQ ID NO: 26; the CDRL2 sequence comprising SEQ ID NO: 27; and the CDRL3 sequence comprising SEQ ID NO: 28; (1) the CDRH1 sequence comprising SEQ ID NO: 35; the CDRH2 sequence comprising SEQ ID NO: 36; the CDRH3 comprising SEQ ID NO: 37; the CDRL1 sequence comprising SEQ ID NO: 38; the CDRL2 sequence comprising SEQ ID NO: 39; and the CDRL3 sequence comprising SEQ ID NO: 40; (m) the CDRH1 sequence comprising SEQ ID NO: 41; the CDRH2 sequence comprising SEQ ID NO: 42; the CDRH3 comprising SEQ ID NO: 43; the CDRL1 sequence comprising SEQ ID NO: 44; the CDRL2 sequence comprising SEQ ID NO: 45; and the CDRL3 sequence comprising SEQ ID NO: 46; (n) the CDRH1 sequence comprising SEQ ID NO: 47; the CDRH2 sequence comprising SEQ ID NO: 48; the CDRH3 comprising SEQ ID NO: 49; the CDRL1 sequence comprising SEQ ID NO: 50; the CDRL2 sequence comprising SEQ ID NO: 51; and the CDRL3 sequence comprising SEQ ID NO: 52; (0) the CDRH1 sequence comprising SEQ ID NO: 59; the CDRH2 sequence comprising SEQ ID NO: 60; the CDRH3 comprising SEQ ID NO: 61; the CDRL1 sequence comprising SEQ ID NO: 62; the CDRL2 sequence comprising SEQ ID NO: 63; and the CDRL3 sequence comprising SEQ ID NO: 64; (p) the CDRH1 sequence comprising SEQ ID NO: 65; the CDRH2 sequence comprising SEQ ID NO: 66; the CDRH3 comprising SEQ ID NO: 67; the CDRL1 sequence comprising SEQ ID NO: 68; the CDRL2 sequence comprising SEQ ID NO: 69; and the CDRL3 sequence comprising SEQ ID NO: 70; (q) the CDRH1 sequence comprising SEQ ID NO: 159; the CDRH2 sequence comprising SEQ ID NO: 160; the CDRH3 comprising SEQ ID NO: 161; the CDRL1 sequence comprising SEQ ID NO: 162; the CDRL2 sequence comprising SEQ ID NO: 163; and the CDRL3 sequence comprising SEQ ID NO: 164; (r) the CDRH1 sequence comprising SEQ ID NO: 135; the CDRH2 sequence comprising SEQ ID NO: 136; the CDRH3 comprising SEQ ID NO: 137; the CDRL1 sequence comprising SEQ ID NO: 138; the CDRL2 sequence comprising SEQ ID NO: 139; and the CDRL3 sequence comprising SEQ ID NO: 140; (s) the CDRH1 sequence comprising SEQ ID NO: 141; the CDRH2 sequence comprising SEQ ID NO: 142; the CDRH3 comprising SEQ ID NO: 143; the CDRL1 sequence comprising SEQ ID NO: 144; the CDRL2 sequence comprising SEQ ID NO: 145; and the CDRL3 sequence comprising SEQ ID NO: 146; or (t) the CDRH1 sequence comprising SEQ ID NO: 147; the CDRH2 sequence comprising SEQ ID NO: 148; the CDRH3 comprising SEQ ID NO: 149; the CDRL1 sequence comprising SEQ ID NO: 150; the CDRL2 sequence comprising SEQ ID NO: 151; and the CDRL3 sequence comprising SEQ ID NO: 152.

In a second aspect, provided herein is a method for depleting mast cells in a subject in need thereof, the method comprising administering to the subject the composition described herein. In embodiments, the subject has mastocytosis, cutaneous mastocytosis, indolent systemic mastocytosis, systemic smoldering mastocytosis, aggressive systemic mastocytosis, mast cell leukemia, mast cell sarcoma, or systemic mastocytosis.

In a third aspect, provided herein is a method for preparing a therapeutic nanoparticle, the method comprising: synthesizing the nanoparticle with a therapeutic compound by: mixing a poly(propylene sulfone) (PPSU) homopolymer and the therapeutic compound in DMSO; adding water to the DMSO; and removing the DMSO by dialysis; and adsorbing a targeting protein onto the surface of the nanoparticle by: incubating the nanoparticle with the targeting protein in a buffer.

In embodiments, the therapeutic compound is midostaurin. In embodiments, the targeting protein is an antibody. In embodiments, the targeting protein is an anti-siglec 6 antibody. In embodiments, the antibody is selected from: anti-siglec-6 antibody clone 767329; and an antibody comprising: a CDRH1 comprising a sequence selected from SEQ ID NOs: 89, 17, 83, 5, 11, 77, 71, 168, 53, 29, 23, 35, 41, 47, 59, 65, 135, 141, 147; a CDRH2 comprising a sequence selected from SEQ ID NOs: 90, 18, 84, 6, 12, 78, 72, 169, 54, 30, 24, 36, 42, 48, 60, 66, 136, 142, 148; a CDRH3 comprising a sequence selected from SEQ ID NOs: 91, 19, 85, 7, 13, 79, 73, 7, 55, 31, 25, 37, 43, 49, 61, 67, 137, 143, 149; a CDRL1 comprising a sequence selected from SEQ ID NOs: 92, 20, 86, 8, 14, 80, 74, 8, 56, 32, 26, 38, 44, 50, 62, 68, 138, 144, 150; a CDRL2 comprising a sequence selected from SEQ ID NOs: 93, 21, 87, 9, 15, 81, 75, 9, 57, 33, 27, 39, 45, 51, 63, 69, 139, 145, 151; and a CDRL3 comprising a sequence selected from SEQ ID NOs: 94, 22, 88, 10, 16, 82, 76, 10, 58, 34, 28, 40, 46, 52, 64, 70, 140, 146, 152.

In embodiments, the anti-siglec 6 antibody comprises: (a) the CDRH1 sequence comprising SEQ ID NO: 89; the CDRH2 sequence comprising SEQ ID NO: 90; the CDRH3 comprising SEQ ID NO: 91; the CDRL1 sequence comprising SEQ ID NO: 92; the CDRL2 sequence comprising SEQ ID NO: 93; and the CDRL3 sequence comprising SEQ ID NO: 94; (b) the CDRH1 sequence comprising SEQ ID NO: 17; the CDRH2 sequence comprising SEQ ID NO: 18; the CDRH3 comprising SEQ ID NO: 19; the CDRL1 sequence comprising SEQ ID NO: 20; the CDRL2 sequence comprising SEQ ID NO: 21; and the CDRL3 sequence comprising SEQ ID NO: 22; (c) the CDRH1 sequence comprising SEQ ID NO: 83; the CDRH2 sequence comprising SEQ ID NO: 84; the CDRH3 comprising SEQ ID NO: 85; the CDRL1 sequence comprising SEQ ID NO: 86; the CDRL2 sequence comprising SEQ ID NO: 87; and the CDRL3 sequence comprising SEQ ID NO: 88; (d) the CDRH1 sequence comprising SEQ ID NO: 5; the CDRH2 sequence comprising SEQ ID NO: 6; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10; (e) the CDRH1 sequence comprising SEQ ID NO: 11; the CDRH2 sequence comprising SEQ ID NO: 12; the CDRH3 comprising SEQ ID NO: 13; the CDRL1 sequence comprising SEQ ID NO: 14; the CDRL2 sequence comprising SEQ ID NO: 15; and the CDRL3 sequence comprising SEQ ID NO: 16; (f) the CDRH1 sequence comprising SEQ ID NO: 77; the CDRH2 sequence comprising SEQ ID NO: 78; the CDRH3 comprising SEQ ID NO: 79; the CDRL1 sequence comprising SEQ ID NO: 80; the CDRL2 sequence comprising SEQ ID NO: 81; and the CDRL3 sequence comprising SEQ ID NO: 82; (g) the CDRH1 sequence comprising SEQ ID NO: 71; the CDRH2 sequence comprising SEQ ID NO: 72; the CDRH3 comprising SEQ ID NO: 73; the CDRL1 sequence comprising SEQ ID NO: 74; the CDRL2 sequence comprising SEQ ID NO: 75; and the CDRL3 sequence comprising SEQ ID NO: 76; (h) the CDRH1 sequence comprising SEQ ID NO: 168; the CDRH2 sequence comprising SEQ ID NO: 169; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10; (i) the CDRH1 sequence comprising SEQ ID NO: 53; the CDRH2 sequence comprising SEQ ID NO: 54; the CDRH3 comprising SEQ ID NO: 55; the CDRL1 sequence comprising SEQ ID NO: 56; the CDRL2 sequence comprising SEQ ID NO: 57; and the CDRL3 sequence comprising SEQ ID NO: 58; (j) the CDRH1 sequence comprising SEQ ID NO: 29; the CDRH2 sequence comprising SEQ ID NO: 30; the CDRH3 comprising SEQ ID NO: 31; the CDRL1 sequence comprising SEQ ID NO: 32; the CDRL2 sequence comprising SEQ ID NO: 33; and the CDRL3 sequence comprising SEQ ID NO: 34; (k) the CDRH1 sequence comprising SEQ ID NO: 23; the CDRH2 sequence comprising SEQ ID NO: 24; the CDRH3 comprising SEQ ID NO: 25; the CDRL1 sequence comprising SEQ ID NO: 26; the CDRL2 sequence comprising SEQ ID NO: 27; and the CDRL3 sequence comprising SEQ ID NO: 28; (l) the CDRH1 sequence comprising SEQ ID NO: 35; the CDRH2 sequence comprising SEQ ID NO: 36; the CDRH3 comprising SEQ ID NO: 37; the CDRL1 sequence comprising SEQ ID NO: 38; the CDRL2 sequence comprising SEQ ID NO: 39; and the CDRL3 sequence comprising SEQ ID NO: 40; (m) the CDRH1 sequence comprising SEQ ID NO: 41; the CDRH2 sequence comprising SEQ ID NO: 42; the CDRH3 comprising SEQ ID NO: 43; the CDRL1 sequence comprising SEQ ID NO: 44; the CDRL2 sequence comprising SEQ ID NO: 45; and the CDRL3 sequence comprising SEQ ID NO: 46; (n) the CDRH1 sequence comprising SEQ ID NO: 47; the CDRH2 sequence comprising SEQ ID NO: 48; the CDRH3 comprising SEQ ID NO: 49; the CDRL1 sequence comprising SEQ ID NO: 50; the CDRL2 sequence comprising SEQ ID NO: 51; and the CDRL3 sequence comprising SEQ ID NO: 52; (0) the CDRH1 sequence comprising SEQ ID NO: 59; the CDRH2 sequence comprising SEQ ID NO: 60; the CDRH3 comprising SEQ ID NO: 61; the CDRL1 sequence comprising SEQ ID NO: 62; the CDRL2 sequence comprising SEQ ID NO: 63; and the CDRL3 sequence comprising SEQ ID NO: 64; (p) the CDRH1 sequence comprising SEQ ID NO: 65; the CDRH2 sequence comprising SEQ ID NO: 66; the CDRH3 comprising SEQ ID NO: 67; the CDRL1 sequence comprising SEQ ID NO: 68; the CDRL2 sequence comprising SEQ ID NO: 69; and the CDRL3 sequence comprising SEQ ID NO: 70; (q) the CDRH1 sequence comprising SEQ ID NO: 159; the CDRH2 sequence comprising SEQ ID NO: 160; the CDRH3 comprising SEQ ID NO: 161; the CDRL1 sequence comprising SEQ ID NO: 162; the CDRL2 sequence comprising SEQ ID NO: 163; and the CDRL3 sequence comprising SEQ ID NO: 164; (r) the CDRH1 sequence comprising SEQ ID NO: 135; the CDRH2 sequence comprising SEQ ID NO: 136; the CDRH3 comprising SEQ ID NO: 137; the CDRL1 sequence comprising SEQ ID NO: 138; the CDRL2 sequence comprising SEQ ID NO: 139; and the CDRL3 sequence comprising SEQ ID NO: 140; (s) the CDRH1 sequence comprising SEQ ID NO: 141; the CDRH2 sequence comprising SEQ ID NO: 142; the CDRH3 comprising SEQ ID NO: 143; the CDRL1 sequence comprising SEQ ID NO: 144; the CDRL2 sequence comprising SEQ ID NO: 145; and the CDRL3 sequence comprising SEQ ID NO: 146; or (t) the CDRH1 sequence comprising SEQ ID NO: 147; the CDRH2 sequence comprising SEQ ID NO: 148; the CDRH3 comprising SEQ ID NO: 149; the CDRL1 sequence comprising SEQ ID NO: 150; the CDRL2 sequence comprising SEQ ID NO: 151; and the CDRL3 sequence comprising SEQ ID NO: 152.

In embodiments, the step of adding water to the DMSO comprises adding the water at a ratio of about 2:1 to about 10:1. In embodiments, the water is added in at least two increments, wherein each increment comprises a ratio of about 2:1 to the DMSO. In embodiments, the therapeutic compound is at between about 1.2 wt % and about 5 wt %. In embodiments, the therapeutic compound is at about 2.5 wt %.

In embodiments, the step of incubating the nanoparticle with the targeting protein in the buffer is done for between about 1 second and about 5 minutes at between about 20° C. and about 22° C. In embodiments, the targeting protein is incubated at a higher concentration than the nanoparticle. In embodiments, the targeting protein is incubated at between about 0.001 wt % and about 1000 wt % PPSU. In embodiments, PPSU homopolymer comprises PPSU₂₀.

In a fourth aspect, provided herein is a therapeutic nanoparticle prepared by the methods described herein.

### BRIEF DESCRIPTION OF THE DRAWINGS

FIGS. 1A-1D. MTT assay analysis of mast cell lines treated with free midostaurin (MdS), anti-Siglec-6 PPSU NPs, and anti-Siglec-6 PPSU NPs with MdS payloads. FIGS. 1A and 1B. Both ROSA cell lines displayed similar reactions to treatment with free drug and targeted drug payloads. FIG. 1C. LUVA cells were highly sensitive to the anti-Siglec-6 PPSU NPs with MdS payload, compared to free drug. FIG. 1D. Primary human skin mast cells were also more sensitive to the nanotherapy than the free MdS, however it was not as dramatic as observed in our micelle formulations. Statistical comparisons utilized two-way ANOVA with multiple comparisons, n=3 (A, C) and n=2 (B, D). **=p<0.01, ***=p<0.005, ****=p<0.001. IC50 values for nanoparticle payloads vs free drug were 0.51 vs 0.68 μg/mL (A), 1.24 vs 1.37 μg/mL (B), 0.41 vs 4.1 μg/mL (C), and 0.66 vs 1.5 μg/mL (D).

FIGS. 2A-2B. Siglec-6 receptor quantification of mast cell lines and primary cells. FIG. 2A. The immortalized cell lines tested revealed relatively low receptor expression. FIG. 2B. Primary cells displayed robust amounts of Siglec-6 receptor even by the time of usage at 6-10 weeks. Samples in (A) were compared via one-way ANOVA with multiple comparisons, n=4. **=p<0.01, ***=p<0.005. Cell lines were measured between passages 10 and 12. Primary cell data borrowed with permission from Robida P*, Rische C H, et al. “Functional and Phernotypic Characterization of Siglec-6 on Human Mast Cells”. Cells, March 2022.

FIGS. 3A-3C. ROSA^{D816V} engrafted NSG-SGM3 mice as a model for mastocytosis. FIG. 3A. Schematic of mouse engraftment and timeline of testing reagents. FIG. 3B. Anti-Siglec-6 Fab fragment conjugated PEG-b-PPS micelles with MdS payloads displayed dramatic potency against ROSA^{D816V} cells in the bone marrow (marked here as hCD45+ cells). FIG. 3C. By comparison, anti-Siglec-6 PPSU NPs with MdS payloads were less effective. However, the micelles incurred more damage to mCD45 cells than the PPSU analogues.

FIGS. 4A-4B. Validation of Siglec-6 expression in CPA3+/Siglec-6+ mouse using flow cytometry. FIG. 4A. This gating strategy is also used to identify intact mast cell populations in peritoneal lavage samples taken from mice treated with experimental groups designed to target and deplete cells via Siglec-6. FIG. 4B. Fluorescence minus-ones (FMOs) are used to help positively identify stained cell populations.

FIG. 5. Flow cytometry analysis of Siglec-6 KI mice treated with anti-Siglec-6 antibody-presenting PPSU NPs with midostaurin (MdS) payloads. The depletion of peritoneal mast cells was robust when treated with the targeted payload, however results were not as consistent as when we tested anti-Siglec-6 micelle-based payloads. Statistical analysis was conducted using two-way ANOVA with multiple comparisons. n=3, *=p<0.05, **=p<0.01.

FIG. 6. Schematic showing addition of monoclonal antibodies (mAbs) to the surface of PPSU NPs containing encapsulated midostaurin.

### DETAILED DESCRIPTION

Disclosed herein is a targeted nanotherapy using propylene sulfone-based nanoparticles that delivers chemotherapeutics, including midostaurin, selectively to mast cells to enhance efficacy, while decreasing toxicity and side effects. As demonstrated in the Examples, vesicular nanoparticles composed of poly(propylene sulfone) encapsulating midostaurin and presenting anti-siglec 6 antibodies on their surfaces, were engineered and employed to selectively target mast cells in a mouse model of mastocytosis. Compared to controls (midostaurin alone without nanocarriers) the nanotherapy decreased off-target toxicity by >90% and enhanced cancer cell killing by ˜50%.

Accordingly, in a first aspect of the invention, a method for preparing a therapeutic nanoparticle comprising a therapeutic compound and a targeting protein is provided. The method comprises synthesizing the nanoparticle with the therapeutic compound and adsorbing a targeting protein onto the surface of the nanoparticle, wherein the nanoparticle comprises a poly(propylene sulfone) (PPSU) homopolymer.

The terms “nanoparticle,” “nanocarrier,” and “nanostructure” are used interchangeably herein to refer to a nanomaterial used as a transport module for another substance. Nanoparticles range in size between about 1 nm to about 200 nm and any size in-between (e.g., between 10 nm and 200 nm, between 1 nm and 100 nm, between 1 nm and 40 nm, between 1 nm and 30 nm, between 1 nm and 20 nm, between 1 nm and 15 nm, between 100 nm and 200 nm, and between 150 nm and 200 nm). The nanoparticles disclosed herein are used as a transport module for one or more therapeutic agents. The nanoparticles provided herein are poly(propylene sulfonate) (PPSU), comprising repeat units of PPSU. PPSU nanoparticles may comprise between 2 and 100,000 repeat units of PPSU. In preferred embodiments, the PPSU nanoparticle comprises between 10 and 40 repeat units of PPSU (e.g. 10, 15, 20, 25, 30, 35, 40, etc.). The PPSU nanoparticle may comprises 20 repeat units of PPSU (PPSU₂₀).

As used herein, the term “therapeutic compound” refers to a chemical compound or pharmaceutically acceptable salt thereof having a therapeutic effect. A therapeutic compound relieves to some extent one or more signs, symptoms, or causes of a disease or condition. In exemplary embodiments, the therapeutic compound relieves the symptoms of a disease or condition characterized by mastocytosis. In further exemplary embodiments, the therapeutic compound is midostaurin. Other therapeutic compounds contemplated for use in the method include, but are not limited to, the known classes of drugs including immunosuppressive agents such as cyclosporins (cyclosporin A), immunoactive agents, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, xanthines, anti-oxidants, preservatives, vitamins, and nutrients. Cargo may also include combinations of, complexes of, mixtures of or other associations of any of the cargo molecules listed. Combinations, complexes and mixtures of cargo may be in the same nanostructure or administered at the same time, but in separate nanostructures.

The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by one of skill in the art that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein may not be critical to the activity of the compound, so long as the salt as a whole is pharmacologically acceptable and as long as the counter-ion does not contribute undesired qualities to the salt as a whole. Undesired qualities may include undesirable solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

As used herein, the term “targeting protein” refers to a protein that binds to a desired molecule or targets a particular cell type. When expressed on the nanoparticle surface, the targeting protein will deliver the therapeutic compound to a location in which the desired molecule is expressed. For example, the targeting protein may selectively bind a cell surface receptor that is preferentially, more abundantly, or exclusively expressed on a particular cell type. The targeting protein may be an antibody. In exemplary embodiments, the targeting protein is an antibody that binds to a receptor expressed on a particular cell type. The targeting protein may be an antibody that binds to a siglec receptor, e.g. a siglec-6 receptor or a siglec-8 receptor. In further exemplary embodiments, the targeting protein is an anti-siglec 6 antibody.

Siglecs (sialic acid-binding immunoglobulin-type lectins) are cell surface receptors that bind sialic acid. They are found primarily on the surface of immune cells and are a subset of the I-type lectins. Engagement of CD33-family sialic acid-binding immunoglobulin-like lectin (Siglec) receptors are shown to have inhibitory effects on various cells including mast cells^{1,2}. Of the siglec family of proteins that are expressed on mast cells (Siglec-2/CD22, Siglec-3/CD33, Siglec-5, Siglec-6, Siglec-8, and Siglec-10), siglec-6 is most specific. Sialic acid-binding Ig-like lectin 6 (Siglec-6) is a protein that is encoded by the SIGLEC6 gene. Siglec-6 is also called CD33-like, OB [leptin]-binding protein 1 (OB-BP1) and CD327. The full-length sequence of Siglec-6 is MQGAQEASASEMLPLLLPLLWAGALAQERRFQLEGPESLTVQEGLCVLVPCRLPTTLPA SYYGYGYWFLEGADVPVATNDPDEEVQEETRGRFHLLWDPRRKNCSLSIRDARRRDNA AYFFRLKSKWMKYGYTSSKLSVRVMALTHRPNISIPGTLESGHPSNLTCSVPWVCEQGT PPIFSWMSAAPTSLGPRTTQSSVLTITPRPQDHSTNLTCQVTFPGAGVTMERTIQLNVSYA PQKVAISIFQGNSAAFKILQNTSSLPVLEGQALRLLCDADGNPPAHLSWFQGFPALNATPI SNTGVLELPQVGSAEEGDFTCRAQHPLGSLQISLSLFVHWKPEGRAGGVLGAVWGASIT TLVFLCVCFIFRVKTRRKKAAQPVQNTDDVNPVMVSGSRGHQHQFQTGIVSDHPAEAG PISEDEQELHYAVLHFHKVQPQEPKVTDTEYSEIKIHK (SEQ ID NO: 1).

The anti-siglec 6 antibody may be clone 767329.

The anti-siglec 6 antibody may be an antibody disclosed in U.S. Publication No. 2024/0084005.

The anti-siglec 6 antibody may comprise a CDRH1 comprising a sequence selected from SEQ ID NOs: 89, 17, 83, 5, 11, 77, 71, 168, 53, 29, 23, 35, 41, 47, 59, 65, 135, 141, 147; a CDRH2 comprising a sequence selected from SEQ ID NOs: 90, 18, 84, 6, 12, 78, 72, 169, 54, 30, 24, 36, 42, 48, 60, 66, 136, 142, 148; a CDRH3 comprising a sequence selected from SEQ ID NOs: 91, 19, 85, 7, 13, 79, 73, 7, 55, 31, 25, 37, 43, 49, 61, 67, 137, 143, 149; a CDRL1 comprising a sequence selected from SEQ ID NOs: 92, 20, 86, 8, 14, 80, 74, 8, 56, 32, 26, 38, 44, 50, 62, 68, 138, 144, 150; a CDRL2 comprising a sequence selected from SEQ ID NOs: 93, 21, 87, 9, 15, 81, 75, 9, 57, 33, 27, 39, 45, 51, 63, 69, 139, 145, 151; and a CDRL3 comprising a sequence selected from SEQ ID NOs: 94, 22, 88, 10, 16, 82, 76, 10, 58, 34, 28, 40, 46, 52, 64, 70, 140, 146, 152. The anti-siglec 6 antibody may comprise:
(a) the CDRH1 sequence comprising SEQ ID NO: 89; the CDRH2 sequence comprising SEQ ID NO: 90; the CDRH3 comprising SEQ ID NO: 91; the CDRL1 sequence comprising SEQ ID NO: 92; the CDRL2 sequence comprising SEQ ID NO: 93; and the CDRL3 sequence comprising SEQ ID NO: 94;
(b) the CDRH1 sequence comprising SEQ ID NO: 17; the CDRH2 sequence comprising SEQ ID NO: 18; the CDRH3 comprising SEQ ID NO: 19; the CDRL1 sequence comprising SEQ ID NO: 20; the CDRL2 sequence comprising SEQ ID NO: 21; and the CDRL3 sequence comprising SEQ ID NO: 22;
(c) the CDRH1 sequence comprising SEQ ID NO: 83; the CDRH2 sequence comprising SEQ ID NO: 84; the CDRH3 comprising SEQ ID NO: 85; the CDRL1 sequence comprising SEQ ID NO: 86; the CDRL2 sequence comprising SEQ ID NO: 87; and the CDRL3 sequence comprising SEQ ID NO: 88;
(d) the CDRH1 sequence comprising SEQ ID NO: 5; the CDRH2 sequence comprising SEQ ID NO: 6; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10;
(c) the CDRH1 sequence comprising SEQ ID NO: 11; the CDRH2 sequence comprising SEQ ID NO: 12; the CDRH3 comprising SEQ ID NO: 13; the CDRL1 sequence comprising SEQ ID NO: 14; the CDRL2 sequence comprising SEQ ID NO: 15; and the CDRL3 sequence comprising SEQ ID NO: 16;
(f) the CDRH1 sequence comprising SEQ ID NO: 77; the CDRH2 sequence comprising SEQ ID NO: 78; the CDRH3 comprising SEQ ID NO: 79; the CDRL1 sequence comprising SEQ ID NO: 80; the CDRL2 sequence comprising SEQ ID NO: 81; and the CDRL3 sequence comprising SEQ ID NO: 82;
(g) the CDRH1 sequence comprising SEQ ID NO: 71; the CDRH2 sequence comprising SEQ ID NO: 72; the CDRH3 comprising SEQ ID NO: 73; the CDRL1 sequence comprising SEQ ID NO: 74; the CDRL2 sequence comprising SEQ ID NO: 75; and the CDRL3 sequence comprising SEQ ID NO: 76;
(h) the CDRH1 sequence comprising SEQ ID NO: 168; the CDRH2 sequence comprising SEQ ID NO: 169; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10;
(i) the CDRH1 sequence comprising SEQ ID NO: 53; the CDRH2 sequence comprising SEQ ID NO: 54; the CDRH3 comprising SEQ ID NO: 55; the CDRL1 sequence comprising SEQ ID NO: 56; the CDRL2 sequence comprising SEQ ID NO: 57; and the CDRL3 sequence comprising SEQ ID NO: 58;
(j) the CDRH1 sequence comprising SEQ ID NO: 29; the CDRH2 sequence comprising SEQ ID NO: 30; the CDRH3 comprising SEQ ID NO: 31; the CDRL1 sequence comprising SEQ ID NO: 32; the CDRL2 sequence comprising SEQ ID NO: 33; and the CDRL3 sequence comprising SEQ ID NO: 34;
(k) the CDRH1 sequence comprising SEQ ID NO: 23; the CDRH2 sequence comprising SEQ ID NO: 24; the CDRH3 comprising SEQ ID NO: 25; the CDRL1 sequence comprising SEQ ID NO: 26; the CDRL2 sequence comprising SEQ ID NO: 27; and the CDRL3 sequence comprising SEQ ID NO: 28;
(l) the CDRH1 sequence comprising SEQ ID NO: 35; the CDRH2 sequence comprising SEQ ID NO: 36; the CDRH3 comprising SEQ ID NO: 37; the CDRL1 sequence comprising SEQ ID NO: 38; the CDRL2 sequence comprising SEQ ID NO: 39; and the CDRL3 sequence comprising SEQ ID NO: 40;
(m) the CDRH1 sequence comprising SEQ ID NO: 41; the CDRH2 sequence comprising SEQ ID NO: 42; the CDRH3 comprising SEQ ID NO: 43; the CDRL1 sequence comprising SEQ ID NO: 44; the CDRL2 sequence comprising SEQ ID NO: 45; and the CDRL3 sequence comprising SEQ ID NO: 46;
(n) the CDRH1 sequence comprising SEQ ID NO: 47; the CDRH2 sequence comprising SEQ ID NO: 48; the CDRH3 comprising SEQ ID NO: 49; the CDRL1 sequence comprising SEQ ID NO: 50; the CDRL2 sequence comprising SEQ ID NO: 51; and the CDRL3 sequence comprising SEQ ID NO: 52;
(o) the CDRH1 sequence comprising SEQ ID NO: 59; the CDRH2 sequence comprising SEQ ID NO: 60; the CDRH3 comprising SEQ ID NO: 61; the CDRL1 sequence comprising SEQ ID NO: 62; the CDRL2 sequence comprising SEQ ID NO: 63; and the CDRL3 sequence comprising SEQ ID NO: 64;
(p) the CDRH1 sequence comprising SEQ ID NO: 65; the CDRH2 sequence comprising SEQ ID NO: 66; the CDRH3 comprising SEQ ID NO: 67; the CDRL1 sequence comprising SEQ ID NO: 68; the CDRL2 sequence comprising SEQ ID NO: 69; and the CDRL3 sequence comprising SEQ ID NO: 70;
(q) the CDRH1 sequence comprising SEQ ID NO: 159; the CDRH2 sequence comprising SEQ ID NO: 160; the CDRH3 comprising SEQ ID NO: 161; the CDRL1 sequence comprising SEQ ID NO: 162; the CDRL2 sequence comprising SEQ ID NO: 163; and the CDRL3 sequence comprising SEQ ID NO: 164;
(r) the CDRH1 sequence comprising SEQ ID NO: 135; the CDRH2 sequence comprising SEQ ID NO: 136; the CDRH3 comprising SEQ ID NO: 137; the CDRL1 sequence comprising SEQ ID NO: 138; the CDRL2 sequence comprising SEQ ID NO: 139; and the CDRL3 sequence comprising SEQ ID NO: 140;
(s) the CDRH1 sequence comprising SEQ ID NO: 141; the CDRH2 sequence comprising SEQ ID NO: 142; the CDRH3 comprising SEQ ID NO: 143; the CDRL1 sequence comprising SEQ ID NO: 144; the CDRL2 sequence comprising SEQ ID NO: 145; and the CDRL3 sequence comprising SEQ ID NO: 146; or
(t) the CDRH1 sequence comprising SEQ ID NO: 147; the CDRH2 sequence comprising SEQ ID NO: 148; the CDRH3 comprising SEQ ID NO: 149; the CDRL1 sequence comprising SEQ ID NO: 150; the CDRL2 sequence comprising SEQ ID NO: 151; and the CDRL3 sequence comprising SEQ ID NO: 152.

The term “antibody” refers to a full-length antibody, derivatives or fragments of full length antibodies that comprise less than the full-length sequence of the antibody but retain at least the binding specificity of the full-length antibody (e.g., variable portions of the light chain and heavy chain), chimeric antibodies, humanized antibodies, synthetic antibodies, recombinantly produced antibodies, as known to those skilled in the art, and produced using methods known in the art. An antibody of a particular IgG class may be referred to by its subclass (e.g., IgG1, IgG2, IgG3, and IgG4). Amino acid sequences are known to those skilled in the art for the Fc portion of antibodies of the respective IgG subclass. Examples of antibody fragments include, but are not limited to, Fab, Fab′, F(ab′)₂, scFv, Fv, dimeric scFv, Fd, and Fd. Fragments may be synthesized or generated by enzymatic cleavage using methods known in the art. Antibodies can also be produced in either prokaryotic or eukaryotic in vitro translation systems using methods known in the art.

As used herein, the term “heavy chain” (VH) or “light chain” (VL) respectively refer to an antibody variable heavy (VH) and variable light (VL) domain consisting of a “framework” region interrupted by the three CDRs. The complementarity-determining regions (CDRs) of an antibody are subregions of the variable chains in antibodies involved in binding specific antigens. An antibody may be referred to by the antibody's CDRs, which is typically six CDRs total. Thus, an antibody may be referred to herein by the antibody's CDRs of the heavy chain (V_{H} CDR 1 (CDRH1), V_{H} CDR 2 (CDRH2), and V_{H} CDR 3 (CDRH3)) and the light chain (VI. CDR 1 (CDRL1), V_{L} CDR 2 (CDRL2), and V_{L} CDR 3 (CDRL3)).

Antibodies herein specifically include “chimeric” antibodies (immunoglobulins), as well as fragments of such antibodies, as long as they exhibit the desired biological activity (U.S. Pat. No. 4,816,567; Morrison et al., Proc. Natl. Acad. Sci. USA 81:6851-6855 (1984); Oi et al., Biotechnologies 4 (3): 214-221 (1986); and Liu et al., Proc. Natl. Acad. Sci. USA 84:3439-43 (1987)).

“Humanized” or “CDR grafted” forms of non-human (e.g., murine) antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. The term “hypervariable region” when used herein refers to the amino acid residues of an antibody which are associated with its binding to antigen. The hypervariable regions encompass the amino acid residues of the “complementarity determining regions” or “CDRs”. In some instances, framework region (FW) residues of the human immunoglobulin are also replaced by corresponding non-human residues (so called “back mutations”). Furthermore, humanized antibodies may be modified to comprise residues which are not found in the recipient antibody or in the donor antibody, in order to further improve antibody properties, such as affinity. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); and Reichmann et al., Nature 332:323-329 (1988).

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The expression “linear antibodies” when used throughout this application refers to the antibodies described in Zapata, et al. Protein Eng. 8 (10): 1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

Variable regions and CDR sequences in an antibody can be identified according to general rules that have been developed in the art, such as, as set out above, for example using the Kabat nomenclature system, or by aligning the sequences against a database of known variable regions. CDRs can be defined using different systems such as, e.g., Kabat, Chothia, Kabat/Chothia, McCallum/Contact, IMGT, Gelfand, Honneger, Martin, North, and AbM.

Antibodies can be made by well-known methods, such as described in Harlow and Lane, Antibodies; A Laboratory Manual, Cold Spring Harbor Laboratory, Cold Spring Harbor, N.Y., (1988). Monoclonal antibodies can be produced by immunizing inbred mice with a peptide antigen. The QSOX1-L antibody producing hybridoma cells disclosed herein may be produced by immunizing mice with the peptide NEQEQPLGQWHLS (SEQ ID NO: 17). The mice may be immunized by the IP or SC route in an amount and at intervals sufficient to elicit an immune response. The mice may receive an initial immunization on day 0 and be rested for about 3 to about 30 weeks. Immunized mice may be given one or more booster immunizations of by the intravenous (IV) or subcutaneous (SC) route. Lymphocytes, from antibody positive mice may be obtained by removing spleens from immunized mice by standard procedures known in the art. Hybridoma cells may be produced by mixing the splenic lymphocytes with an appropriate fusion partner under conditions which will allow the formation of stable hybridomas. The antibody producing cells and fusion partner cells may be fused in polyethylene glycol at concentrations from about 30% to about 50%. Fused hybridoma cells may be selected by growth in hypoxanthine, thymidine and aminopterin supplemented Dulbecco's Modified Eagles Medium (DMEM) by procedures known in the art. Supernatant fluids may be collected from growth-positive wells and screened for antibody production by an immunoassay such as an Enzyme Linked Immunosorbent Assay. Hybridoma cells from antibody positive wells may be cloned by a technique such as the soft agar technique of MacPherson, Soft Agar Techniques or by limiting dilution in which hybridomas are diluted in suitable growth media and re-plated such that ˜1 hybridoma cell is pipetted into a single well of a 96-well plate where it grows over the course of 10 days as a single clone, in Tissue Culture Methods and Applications, Kruse and Paterson, Eds., Academic Press, 1973.

The step of synthesizing the nanoparticle with the therapeutic compound may comprise mixing the PPSU homopolymer and the therapeutic compound in DMSO, adding water at a ratio of about 2:1 to about 10:1 to the DMSO, and removing the DMSO by dialysis. The PPSU may be dissolved in DMSO at about 5 mg/ml to about 100 mg/ml, preferably about 25 mg/ml to about 50 mg/ml. Mixing the water at varying ratios (e.g., 2:1 to 10:1 by volume) with the solution of DMSO/PPSU allows for the formation of differing nanostructures depending on the ratio and the method of mixing of the water. The ratio of water to DMSO solution include between 2:1 and 10:1, including ranges in between (e.g., about 2.5:1, 3:1, 4:1, 5:1, 6:1, 7:1, 7.5:1, 8:1, 9:1, 10:1, etc.). Nanostructures formed include, for example, nanobundles (i.e., bundle-like nanogels) and vesicle-like nanogels (nanovesicles). A vesicle-like nanostructure may have a diameter of between 250-50 nm, or any diameter or range in between, e.g. 240 nm, 230 nm, 220 nm, 210 nm, 200 nm, 175 nm, 150 nm, 125 nm, 120 nm, 110 nm, 100 nm, 90 nm, 80 nm, 70 nm, 60 nm, 50 nm, 40 nm, 30 nm, 20 nm. Ranges between 100-20 nm, between 90-10 nm, between 80-15 nm, between 70-20 nm, etc. are contemplated therein. The average size is on a weight basis and is measured by light scattering, microscopy, or other appropriate methods.

The PPSU nanoparticles are able to carry both aqueous (e.g., water soluble) cargo or payloads (therapeutic compounds) or hydrophobic cargo or payloads, depending on if the cargo is added to the water or the DMSO solution before mixing. The therapeutic compounds described herein are mixed in the DMSO, but other therapeutic compounds may be added to the water. Varying the ratio of water to DMSO:PPSU solution and the stepwise addition of the water, e.g., adding the water in 1 to 100 increments, preferably 1 to 80 increments, controls the morphology and size of the nanostructures produced as detailed in U.S. Pat. No. 20210030690A1, which is incorporated herein by reference. In exemplary embodiments, the water is added in two increments, wherein each increment comprises a volumetric ration of about 2:1 to the DMSO, ideally with a final volume of 600 μL or less. Ideal incremental volumes of water include 100 μL×4, 133 μL×3, or 200 μL×2 versus 200 μL of DMSO for the generation of the PPSU vesicles. For micelle PPSU morphologies, ideal incremental volumes of water include 800 μL×1 to 200 μL of DMSO. For bundle PPSU morphologies, ideal incremental volumes of water include 5 μL×80 thru 20 μL×20 to 200 μL of DMSO. The water:DMSO solution comprising the PPSU nanostructures can further undergo dialysis in order to remove the remaining DMSO and produce an aqueous solution that can be administered to a subject. Suitable aqueous solutions include water, phosphate buffer saline (PBS), saline (>3% salt), water or PBS with miscible organics (ethanol, acetone, tetrahydrofuran, DMF, etc), and cell culture medium with or without serum or added miscible organics (including but not limited to DMEM, Dulbecco's, custom serum-free medium, IMDM, RPMI, Hank's balanced salt solution, MEM, etc.).

The PPSU nanoparticle may be loaded with midostaurin (MdS) with a loading efficiency of at least 75%, more preferably at least 80%, still more preferably at least 90%, preferably at least 95%, more preferably at least 98%, and in some instances with a loading efficiency of about 100%. The loading capacity of the PPSU nanostructures may be greater than 75%, preferably at least 80%. The loading efficiency is measured as the ratio of the therapeutic compound (or the targeting protein) encapsulated by the nanoparticle to the total amount of therapeutic compound (or targeting protein) available for loading in the initial solution. It is also envisioned that an additional cargo can be added to both the DMSO solution and the water. In embodiments, midostaurin and additional agents may be loaded into the nanoparticles.

The therapeutic compound may be provided to the DMSO or water at between about 1 wt % and about 6 wt %, or any weight percentage or range in between (e.g. 1.2 wt %, 1.5 wt %, 2 wt %, 3 wt %, 4 wt %, etc.). In exemplary embodiments, midostaurin is added to the DMSO at 1.2 wt % or 2.5 wt %.

The step of adsorbing the targeting protein to the nanoparticle may comprise incubating the nanoparticle with the targeting protein in a buffer for between about 1 second and about 5 minutes at between about 20° C. and about 22° C. Methods for loading PPSU nanoparticles are described in Du et al. (2020). Homopolymer self-assembly of poly(propylene sulfone) hydrogels via dynamic noncovalent sulfone-sulfone bonding. Nature communications 11, 4896; and U.S. Publication No. 20240091164A1 each of which are incorporated by reference herein in their entireties. In embodiments, more than one targeting protein may be added to the nanoparticle surface.

The loading efficiency of the targeting protein(s) on the nanoparticle may be greater than 75%, preferably greater than 80%, more preferably greater than 90%, even more preferably at least 95%.

The targeting protein(s) may be incubated at between about 0.001 wt % and about 1000 wt %, and any wt % or range in between, of the total mass of the PPSU nanoparticle. The targeting protein(s) may be incubated at between about 0.01 wt % and about 5 wt % of the total mass of the PPSU nanoparticle. The concentration of the targeting protein(s) may be higher than the concentration of the nanoparticle. The concentration of the targeting protein(s) may be at least twice as high as the concentration of the nanoparticle in the buffer. Contact between the targeting proteins and PPSU nanoparticles results in decoration of targeting proteins on PPSU nanoparticle surfaces due to physical and electrostatic interactions without the need for covalent binding.

The method may further comprise washing the nanoparticle with the buffer two remove excess protein. The nanoparticle may be washed more than one time. The nanoparticle may be washed between 1 and 3 times.

Nanoparticles can include a polymer coating (e.g., a polymer coating containing dextran) and a nucleic acid or protein linked to the nanoparticle. The targeting protein may be added to the nanoparticle along with a capping or filler agent, such as albumin or other inert plasma protein. In embodiments, the capping agent is bovine serum albumin (BSA) or human albumin. The nanoparticle may possess a negatively charged surface that promotes attachment of positively charged regions of proteins to the nanoparticle. In some embodiments, the surface zeta potential in water of the nanoparticle is in the range of about −100 mV to about +100 mV, and all values in-between, including about 45 mV.

In a second aspect, provided herein is a therapeutic PPSU nanoparticle comprising a therapeutic compound in the PPSU nanogel, and a targeting protein on the nanoparticle surface. The therapeutic nanoparticle may be prepared by any of the methods disclosed herein. A therapeutic PPSU nanoparticle comprising midostaurin and anti-siglec 6 antibodies is designed to target mast cells through antibody binding to the siglec 6 receptor.

In a third aspect, provided herein is a composition for depleting mast cells, the composition comprising a PPSU nanoparticle loaded with midostaurin and coated with an anti-siglec 6 antibody, and a pharmaceutically acceptable carrier, excipient, or diluent. The anti-siglec 6 antibody may be antibody clone 767329.

The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutically acceptable carriers are well known to those skilled in the art and include, but are not limited to, 0.01 to 0.1 M and preferably 0.05 M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders. Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects. A lubricant can be used in a tablet formulation to prevent the tablet and punches from sticking in the die. The lubricant can be chosen from such slippery solids as talc, magnesium and calcium stearate, stearic acid, and hydrogenated vegetable oils. Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compositions. Transdermal patches can include a resinous composition in which a compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

In a fourth aspect, provided herein is a method for depleting mast cells in a subject in need thereof, the method comprising administering to the subject a therapeutic PPSU nanoparticle loaded with midostaurin and coated with an anti-siglec 6 antibody, or a composition comprising the therapeutic nanoparticle.

As used herein, the term “depleting” refers to reducing mast cells by at least 75% and up to 100%.

The term “subject” or “patient” are used herein interchangeably to refer to a mammal, preferably a human, to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. The term “subject” does not denote a particular age or sex. Preferably, the subject is a human. In some embodiments, the subject is a mammal in need of a therapeutic to reduce mast cells. The mast cells may be malignant mast cells. The subject may suffer from mastocytosis, a disorder of abnormal mast cell proliferation, with clinical features that include flushing, pruritis, abdominal pain, diarrhea, hypotension, syncope, and musculoskeletal pain. The subject may suffer from cutaneous mastocytosis, indolent systemic mastocytosis, systemic smoldering mastocytosis, aggressive systemic mastocytosis, mast cell leukemia, mast cell sarcoma, or systemic mastocytosis, or any other subclassification of mastocytosis.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a cell or subject by any suitable route for delivery of the therapeutic agent to the desired location. The therapeutic nanoparticle may be administered via a number of means including, but not limited to, orally, rectally, parenterally (intravenous, intramuscular, or subcutaneous), intracisternally, intravaginally, intraperitoneally, locally (in the form of powders, ointments or drops) or as a buccal or nasal spray. In exemplary embodiments, the therapeutic nanoparticle is administered orally, intravenously, or intraperitoneally.

The disclosed nanoparticles and compositions can be administered as the sole active agent or in combination with other pharmaceutical agents such as other agents used in the treatment of genetic disease in a subject. The amount of the disclosed nanoparticles and compositions comprising the same to be administered is dependent on a variety of factors, including the severity of the condition, the age, sex, and weight of the subject, the frequency of administration, the duration of treatment, and the like. The disclosed nanoparticles and compositions may be administered at any suitable frequency necessary to achieve the desired therapeutic effect, i.e., to treat mastocytosis. The disclosed nanoparticles and compositions may be administered once per day or multiple times per day, once per week for at least 2 weeks. The disclosed nanoparticles and compositions may be administered daily, every other day, every three days, every four days, every five days, every six days, once per week, once every two weeks, or less than once every two weeks. The nanoparticles and compositions may be administered for any suitable duration to achieve the desired therapeutic effect, i.e., treat mastocytosis. For example, the nanocarriers or pharmaceutical compositions may be administered to the subject for one day, two days, three days, four days, five days, six days, seven days, eight days, nine days, ten days, eleven days, twelve days, thirteen days, two weeks, one month, two months, three months, six months, 1 year, or more than 1 year. Any suitable dose of the disclosed nanoparticles and compositions may be used. Suitable doses will depend on the therapeutic compound, intended therapeutic effect, body weight of the individual, age of the individual, and the like. In general, suitable dosages of the disclosed nanocarriers or pharmaceutical compositions comprising the same may range from about 0.025 mg nanocarrier/kg body weight to 200 mg nanocarrier/kg body weight. For example, suitable dosages may be about 0.025 mg/kg, or 0.03 mg/kg, or 0.05 mg/kg, or 0.10 mg/kg, or 0.15 mg/kg, or 0.30 mg/kg, to 0.5 mg/kg, or 0.75 mg/kg, or 1.0 mg/kg, or 1.25 mg/kg, or 1.5 mg/kg, or 1.75 mg/kg, or 2.0 mg/kg. In some embodiments, the suitable doses may be 1 mg nanocarrier/kg body weight, or 3 mg/kg, or 5 mg/kg, or 10 mg/kg, or 25 mg/kg, or 50 mg/kg, or 75 mg/kg, or 100 mg/kg, or 125 mg/kg, or 150 mg/kg, or 175 mg/kg, or 200 mg/kg.

The nanoparticles and compositions can be post processed to yield a sterile aqueous or non-aqueous solution or dispersion or could be isolated, such as via lyophilization and autoclaving, to yield a sterile powder for reconstitution into sterile injectable solutions or dispersions. The nanostructures can be combined with other acceptable compounds for parenteral injection such as but not limited to one or more of the following: water, ethanol, propyleneglycol, polyethyleneglycol, glycerol, vegetable oils, and ethyl oleate. Supplemental additives suitable for parenteral injection can also be used to tailor the composition to that suitable for a specific purpose.

The nanoparticles and compositions may be formulated into a solid dosage form for oral administration such as capsules, tablets, pills, powders, and granules, or the like. In such solid dosage forms, the composition is admixed with one or more supplemental additives falling into the following classes such as, but not limited to, lubricants, buffering agents, wetting agents, adsorption, inert excipients, binders, disintegrating agents, solution retarders, accelerators, adsorbents, or fillers or extenders or other components commonly used by those skilled in the art for production of solid dosage forms.

The nanoparticles and compositions may be formulated into a pharmaceutically acceptable liquid dosage form for oral administration such as a syrup, solution, emulsion, suspension, or elixir. The liquid dosage forms may further comprise inert diluents, solubilizing agents, oils, emulsifiers, adjuvants suspending agents, sweeteners, wetting agents, flavoring agents, perfuming agents or other compounds commonly used by those skilled in the art.

The nanoparticles and compositions may be formulated into a pharmaceutically acceptable liquid dosage form for intravenous administration such that it is a biologically compatible aqueous solution at relevant salt concentration, composition, pH, and temperature. The liquid dosage forms may further comprise inert diluents, solubilizing agents, oils, emulsifiers, adjuvants suspending agents, wetting agents or other compounds commonly used by those skilled in the art.

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids. A protein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The terms encompass amino acid polymers in which one or more amino acid residues is an artificial chemical analog of a corresponding naturally occurring amino acid, as well as to naturally occurring amino acid polymers. The terms polypeptide, peptide, and protein are also inclusive of modifications including, but not limited to, glycosylation, lipid attachment, sulfation, carboxylation, hydroxylation, ADP-ribosylation, and addition of other complex polysaccharides. The terms “residue” or “amino acid residue” or “amino acid” are used interchangeably to refer to an amino acid that is incorporated into a peptide, protein, or polypeptide. The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine(S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine(S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases. The comparison can be based on structural comparison tools integrated into common structure analysis software suites and servers such as PyMol, UCSF ChimeraX, DALI, and HHPRED.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant,” “mutant,” or “derivative” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

### Miscellaneous

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter. The terms “consisting essentially of” and “consisting of” should be interpreted in line with the MPEP and relevant Federal Circuit interpretation.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### EXAMPLES

The following Examples are illustrative and should not be interpreted to limit the scope of the claimed subject matter. Indeed, various modifications of the invention in addition to those shown and described herein will become apparent to those skilled in the art from the foregoing description and the following examples and fall within the scope of the appended claims.

### Example 1. Poly(Propylene Sulfone) Nanoparticles as Targeted Drug Carriers

Characterization of basic poly(propylene sulfone) nanoparticles (PPSU NPs) with protein was first described in the work of Du et al. In brief, drug-loaded PPSU NPs were synthesized by incremental addition of water (200 μL×2 additions) to polymer and drug (2.5 wt % midostaurin) dissolved in 200 μL of DMSO. Particles were then dialyzed to remove DMSO. Dynamic light scattering was used to determine an average particle diameter of 110 (PDI=0.170) before protein loading and 121 (PDI=0.211) after incorporating monoclonal anti-Siglec-6 antibody (R&D Systems, Cat. #MAB2859) and albumin. We collected supernatants during purification of the nanoparticles and measured protein concentration in both the purified, drug-free particle stocks and the supernatant at 280 nm. We chose to use human albumin as a capping agent to cover any unused surface on the PPSU NPs. The intent was that this would decrease non-specific protein interactions where the vesicle surface would interact with proteins on cell surfaces, in biological fluids, or in culture medium. Other inert plasma proteins would make good candidates for this role as well. We used a FITC-labeled albumin to do a rough quantification of albumin incorporation after loading anti-Siglec-6 onto PPSU NPs. We discovered that surface incorporation of fluorescently labeled proteins onto PPSU can slightly shift the emission spectra, however they were still quantifiable if a spectrum scan is conducted to identify the new peak, and a standard curve is generated using PPSU vesicles with known concentrations of adsorbed protein.

We verified antibody loading and conducted dynamic light scattering analysis to ensure nanoparticles were consistent with past work. The loading of midostaurin payloads was verified using UV-Vis spectroscopy techniques to measure eluents (after removal of particles by centrifugation) at 280 nm, prior to loading protein. We found at least 90% of midostaurin was loaded into PPSU when using 2.5 wt % of the drug in ratio to the amount of polymer. Higher weight percentages (such as 5 wt %) were usable as well, however efficiency began to drop and particle polydispersity began to increase, so we chose to conserve material by using 2.5 wt % in these experiments.

It is important to note that while 2.5 wt % loads can result in good quality batches of PPSU NPs with low polydispersity and diameters around 110 nm, the formulations appear to be dependent on the batch quality of midostaurin and having maximally oxidized PPSU polymer. Particle sizes were inconsistent between different drug lots at 5 wt %, sometimes resulting in large particles or aggregates (˜700 nm) upon formulation. The extremely hydrophobic nature of midostaurin may be driving these difficulties, as it is only marginally soluble in DMSO, and a fast rate of water addition might cause the rapid formation of aggregates during nanoparticle nucleation but before PPSU has sufficient time to encapsulate the growing nuclei of drug cores. A simple solution to these issues appears to be decreasing the wt % to 1.25 wt % or less, or modifying the water-addition ratios during formulation.

### In Vitro and In Vivo Experiments

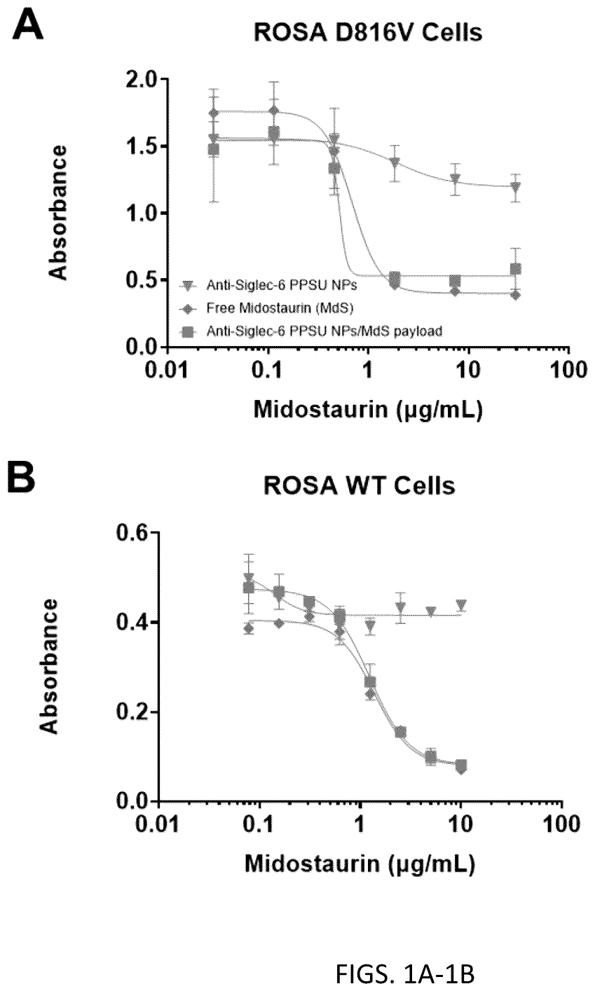

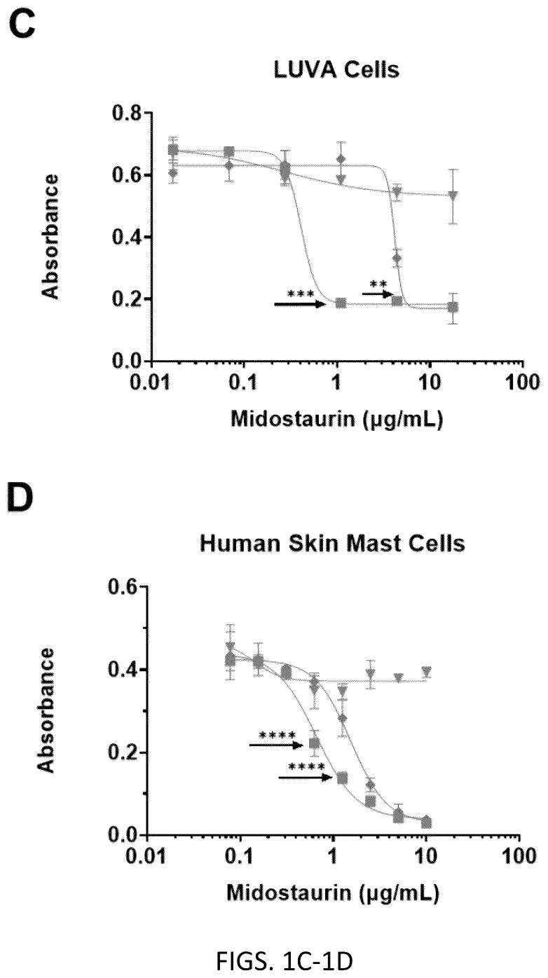

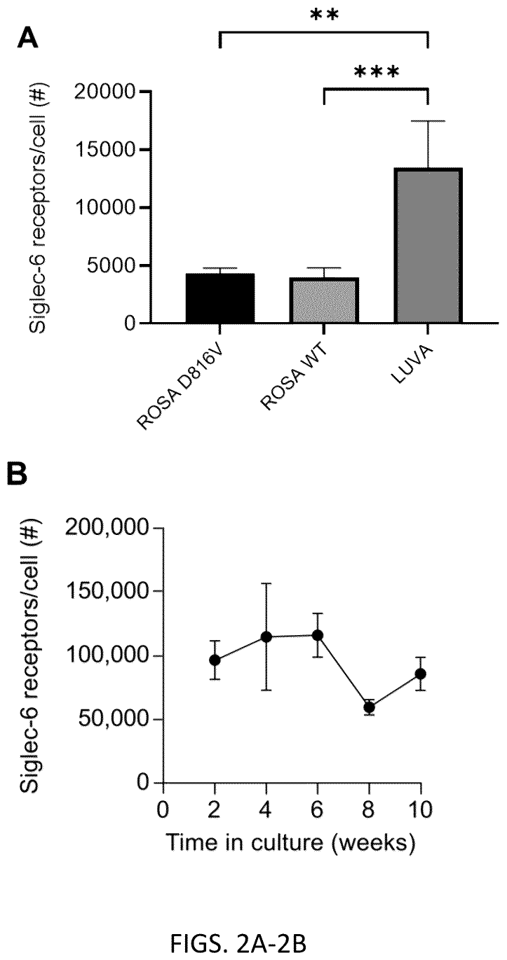

Anti-Siglec-6 PPSU NPs with MdS payloads demonstrated comparable or enhanced potency against different mast cell lines for its effects on reducing cell viability (FIG. 1). In the ROSA human mast cell lines (FIGS. 1A and 1B), the anti-Siglec-6 PPSU NPs with MdS performed comparably to free MdS, regardless of the presence of the D816V mutation (frequent mutation in systemic mastocytosis). However, the nanotherapy showed a robust, ˜10-fold increase in potency when used to treat LUVA human mast cells (FIG. 1C). As with the micelle-based nanotherapy, this may be due to enhanced cell surface interactions resulting from greater cell-surface receptor density. Human primary mast cells were also more sensitive to the nanotherapy with respect to undergoing cell death, with greater reductions in viability at lower concentrations, and compared to free midostaurin, as well as greater maximum cell death compared to the mast cell lines. (FIG. 1D). It appears that the responsiveness of the cell lines might be largely attributed to Siglec-6 receptor density (FIG. 2).

### Murine Models for Studying Mast Cell Depletion

### ROSA^{D816V} Engrafted NSG-SGM3 Mice

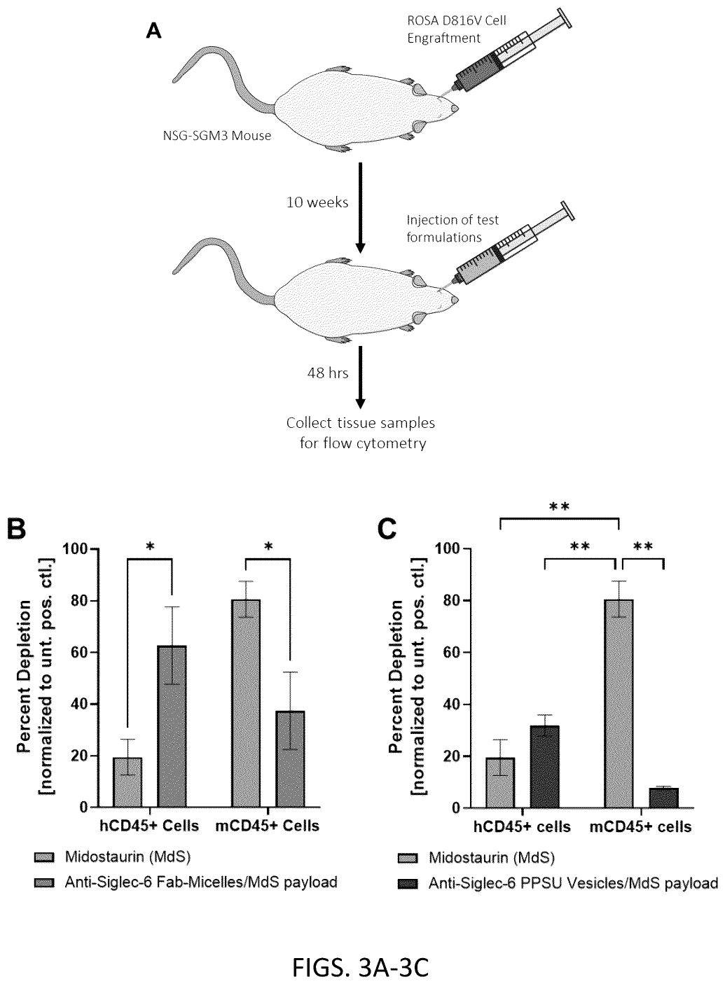

The mice used for engraftment of the ROSA^{D816V} cells are the same strain we utilize for engrafting human CD34+ cells for allergy studies as they produce cytokines that help to maintain mast cells (FIG. 3A). However, we ran into a complication after attempting to adapt previously published methods that employed retroorbital injections to introduce cells. We discovered that localized tumors sometimes develop near the eye around the injection site, requiring premature sacrificing of such animals for humane purposes. A tail vein injection strategy is likely to be a better choice despite the likelihood that there is a lower success rate (e.g., less control over where tumors will develop), and local, inadvertent injection site tumors could be excised from the tail without much consequence for the mouse. Alternatively, subcutaneous injections can most reliably produce localized solid tumors, and may be the most humane option to pursue, despite the differences from typical systemic mastocytosis.

Of the mice we successfully tested (i.e., those without retroorbital tumors), we were able to produce a small amount of pilot data to guide future experimental decisions. Within this experiment, we compared sdFab-micelles loaded with midostaurin, anti-Siglec-6 mAb PPSU NPs loaded with midostaurin, free midostaurin, and unloaded versions of the sdFab-micelles and mAb-vesicles, with a PBS control. We dosed mice based on an approximation of the total peak blood concentration of midostaurin and its active metabolites in humans (2.16 μg/mL). This number worked out to be 3.132 μg of drug per 20 g mouse for an equivalent intravenous dose. This was based on an assumed blood volume of 72 mL/kg of blood per 20 g mouse (taken from the NIH's ‘Guidelines for Survival Bleeding of Mice and Rats’). Because PPSU nanoparticles cannot be delivered orally, intravenous injections were necessary for these experiments. Thus, we utilized midostaurin concentrations of roughly 2.09 μg/mL for each injection. After injection, samples were collected 48 hours later as we expected drug effects to be comparable within a similar timeframe to our in vitro experiments and wanted the best chance of obtaining an observable result.

It is anticipated that the bioavailability and blood concentration of free drug vs encapsulated drug differs drastically, and it may be impossible to create a fair direct comparison without using orally administered midostaurin that results in delivery of the drug and its active metabolites. However, the overall kinetics of this approach would be significantly different than the use of injected nanoparticle payloads which cannot be administered orally. The drug-loaded anti-Siglec-6 PPSU vesicles reduced ROSA D816 burden by about 30% (about 10% greater than free drug) (FIGS. 3A-3C). In contrast, free drug reduced host mCD45+ cells by about 80%, whereas the targeted therapy only reduced the population by about 10%. This massive difference in off-target toxicity indicates that the overall dose of encapsulated drug can likely be increased, and that side effects could be significantly reduced.

When we conducted the same experiment for the PPSU NPs, we observed interesting differences, some of which may be attributable to our small sample size. First, the efficacy of the mAb-vesicles with midostaurin payloads was not as high as the micelle counterpart (30% depletion) however the off-target toxicity was further reduced (down to ˜10% depletion), which may be due to the greater stability of the vesicle structure and its ability to hold its payload.

PPSU has essentially irreversible interactions with proteins, and it is likely that drug payloads are very stable as well. Our data indicates that the drug is arriving at its target one way or another. However, if off-target toxicity is reduced while maintaining at least comparable drug activity (if not slightly better), then this platform has great appeal. Further studies to determine the kinetics and mechanisms of PPSU drug release are essential.

### Siglec-6 Knock-In Mice

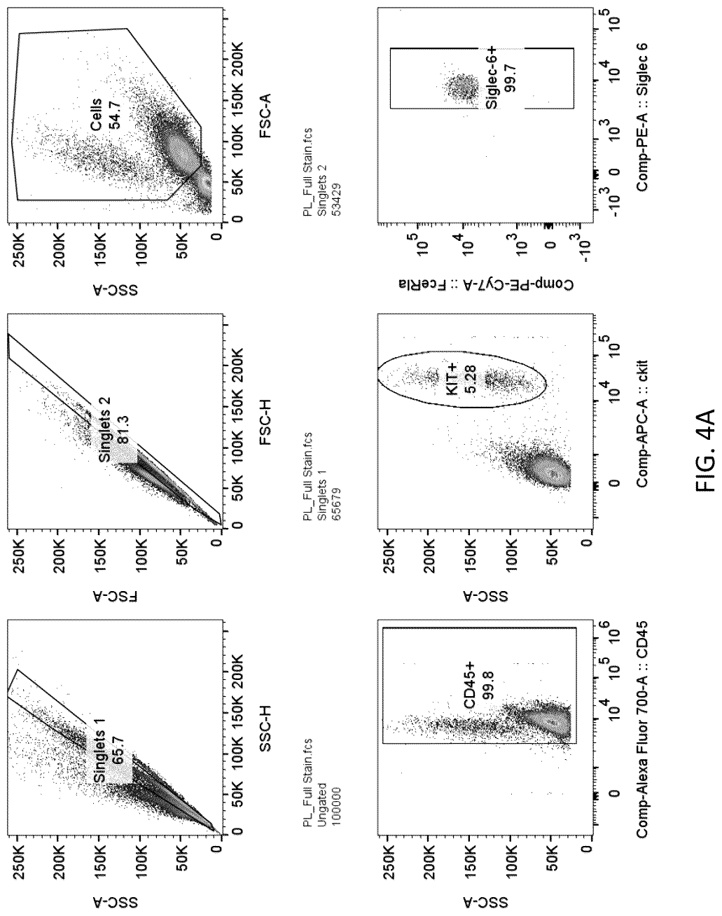

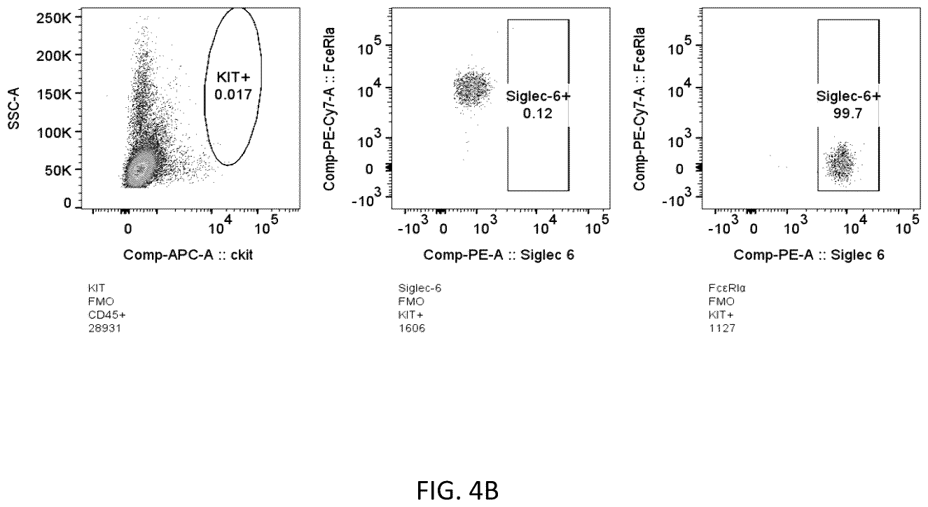

The objective of these experiments was to determine the selectivity of sdFab-micelles for a normal mast cell population, albeit mouse mast cells. We utilized Siglec-6 KI mice, which were ideal test subjects due to their controlled expression of Siglec-6 on transgenically modified mast cells (FIG. 4). In addition, there was no risk of malignant disease complications in the model, as was encountered with the ROSA^{D816V} engrafted mice. This also allows us to evaluate Siglec-6 targeted depletion without convolution that might arise from variations in mast cell engraftment or from using a lentivirally modified cell line like ROSA^{D816V}. We utilized similar methods to the ROSA mouse experiment, with the same dose levels and time points. However, in this experiment, we used intraperitoneal injections, as we were focusing on peritoneal lavage samples to evaluate drug effect, and wanted to ensure maximum exposure.

We tested 3 experimental groups and 2 control groups as follows: anti-Siglec-6 PPSU vesicles with midostaurin payloads, anti-Siglec-6 PPSU vesicles, and PPSU vesicles with midostaurin payloads. The control conditions were free midostaurin and PBS vehicle. We elected to use intraperitoneal injections as our delivery route in this model, as it had a better likelihood of impacting the tissue compartment that contained the largest amount of easily accessible mast cells. To reflect what we did in the ROSA^{D816V} engrafted mice, we collected samples after 48 hours.

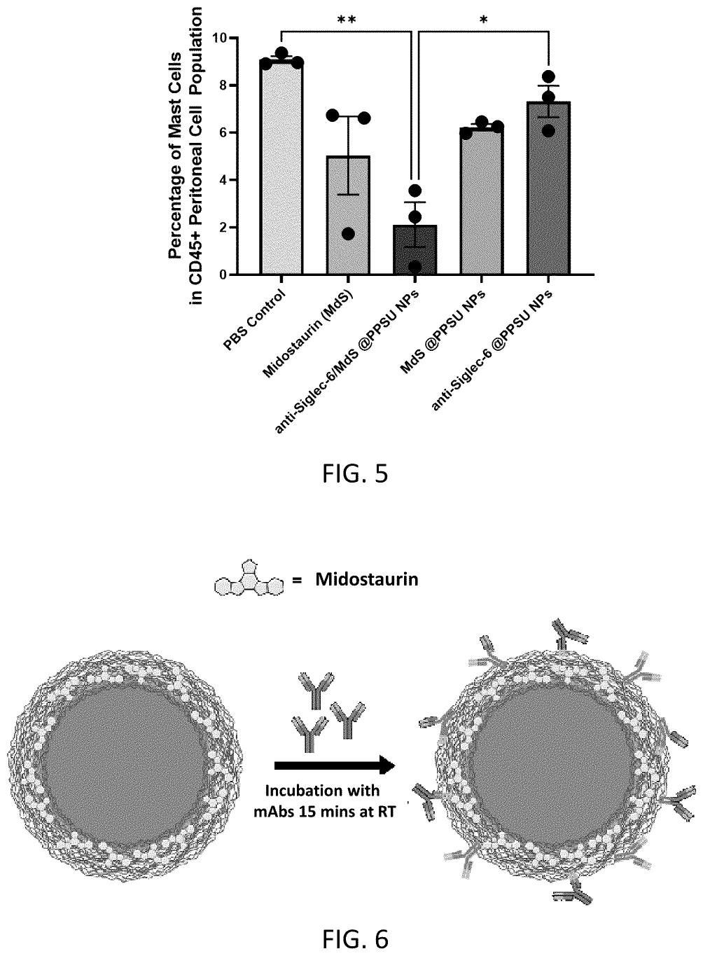

We investigated the use of PPSU NPs as midostaurin delivery vehicles, and found that they depleted mast cells most effectively when presenting adsorbed anti-Siglec-6 (FIG. 5).

TABLE 1
Sequences

| SEQ | |
|---|---|
| ID | |
| NO: | Sequence: |
| 1 | MQGAQEASASEMLPLLLPLLWAGALAQERRFQLEGPESLTVQEGL |
| | CVLVPCRLPTTLPASYYGYGYWFLEGADVPVATNDPDEEVQEETR |
| | GRFHLLWDPRRKNCSLSIRDARRRDNAAYFFRLKSKWMKYGYTSS |
| | KLSVRVMALTHRPNISIPGTLESGHPSNLTCSVPWVCEQGTPPIF |
| | SWMSAAPTSLGPRTTQSSVLTITPRPQDHSTNLTCQVTFPGAGVT |
| | MERTIQLNVSYAPQKVAISIFQGNSAAFKILQNTSSLPVLEGQAL |
| | RLLCDADGNPPAHLSWFQGFPALNATPISNTGVLELPQVGSAEEG |
| | DFTCRAQHPLGSLQISLSLFVHWKPEGRAGGVLGAVWGASITTLV |
| | FLCVCFIFRVKTRRKKAAQPVQNTDDVNPVMVSGSRGHQHQFQTG |
| | IVSDHPAEAGPISEDEQELHYAVLHFHKVQPQEPKVTDTEYSEIK |
| | IHK |
| 5 | GFSLTSYGVS |
| 6 | VIWHDGSTSYHSTLIS |
| 7 | DGYSGTFAY |
| 8 | TASSSVSSSYLH |
| 9 | STSILAS |
| 10 | HQYHRSPYT |
| 11 | GYIFTSYWMY |
| 12 | NVNPYSGDTNYNEKFKT |
| 13 | GDGLYYYAMDY |
| 14 | KASQDINSYLT |
| 15 | RASRLVD |
| 16 | LQYDEFPLT |
| 17 | GYTFTNYYIH |
| 18 | MIHPISGSTNYNEKLKI |
| 19 | RYYGSSYWYFDV |
| 20 | RASSSVSYMN |
| 21 | ATSNLAS |
| 22 | QQWSSHPPT |
| 23 | GYTFTTYGMS |
| 24 | WINTYSGVPTYADDFKG |
| 25 | LAYFGYYFDF |
| 26 | KASQNVGSAVA |
| 27 | SASNRYT |
| 28 | QQYSSYPYT |
| 29 | GFSFSTYVMS |
| 30 | SISGGGSNTYYPDSVKG |
| 31 | REVVVYWYFDV |
| 32 | RASENIYSNLA |
| 33 | SAKNLAD |
| 34 | QHFWGTPLT |
| 35 | GFSLTSYGVS |
| 36 | IIWGDGSTNYHSALIS |
| 37 | GLSDWYFDV |
| 38 | RASSSVSYMH |
| 39 | ATSNLAS |
| 40 | QQWSSNPYT |
| 41 | GYTFTTYGMS |
| 42 | WINTYSGVPTYADDFKG |
| 43 | LAYFGYYFDF |
| 44 | KASQNVGSAVT |
| 45 | SASNRYT |
| 46 | QQYSSYPYT |
| 47 | GFSLTSYGVH |
| 48 | VIWGGGSTDYNAGFIS |
| 49 | ALSDWYFDV |
| 50 | RASSSVSYMH |
| 51 | ATSNLAS |
| 52 | QQWSSNPLT |
| 53 | GYTFTDYYMN |
| 54 | DINPKIGDTIYNQKFKG |
| 55 | WDYYGSSLFDY |
| 56 | KASQDVTTAVA |
| 57 | WASTRHT |
| 58 | QQHYSTPYT |
| 59 | GYTLTTYGMS |
| 60 | WINTYSGVPTYADEFKG |
| 61 | LGYYGYYFDY |
| 62 | RASQSIRNNLH |
| 63 | YIFQSIS |
| 64 | QQSDSWPHVT |
| 65 | GFSLTSYGVN |
| 66 | VIWGDGSTNYHSALIS |
| 67 | ALSDWYFDV |
| 68 | RASSSVSYMH |
| 69 | ATSNLAS |
| 70 | QQWSSNPYT |
| 71 | GYTFTDYKMH |
| 72 | NINPNNGGTSYNQKFKG |
| 73 | RYYGSSSSYFDY |
| 74 | SVSSSVSYMH |
| 75 | DTSKLAS |
| 76 | QQWSSNPPT |
| 77 | GFTFSDYGIH |
| 78 | YITSVSSTIYYADTVKG |
| 79 | LDWYFDV |
| 80 | SANSSVSFMH |
| 81 | DTSKLAS |
| 82 | QQWSSNPLT |
| 83 | GYTFTSYWMH |
| 84 | TIYPGNSDSTYNQKFKG |
| 85 | EGTVVANYYGLDY |
| 86 | RASSSVSYIH |
| 87 | ATSNLAS |
| 88 | QQWSSKPPT |
| 89 | GFSLTSYSVH |
| 90 | VIWSGGSTDYNAVFIS |
| 91 | YSYYTMDY |
| 92 | RSSQSLVLDNGNTYLH |
| 93 | KVSNRFS |
| 94 | SQSTHVPFT |
| 135 | GFNIKDDYMH |
| 136 | WIDLENGDTEYASKFQG |
| 137 | PIGTWAEGFAY |
| 138 | RASENIYSNLA |
| 139 | AATNLAD |
| 140 | QHFWGTPYT |
| 141 | GYTFTDYKMH |
| 142 | NINPNNGGTSYNQKFKG |
| 143 | RYYGSSSSYFDY |
| 144 | RASQSIGTSIH |
| 145 | YASESIS |
| 146 | QQSNSWPTT |
| 147 | GFTFSDYGMH |
| 148 | YISSDSSTIYYADTVKG |
| 149 | TIYYGNYFDY |
| 150 | SASSSVSSSSLY |
| 151 | SMSNLAS |
| 152 | QQWSSNPLT |
| 159 | QVQLQESGPGLVKPSETLSLTCTVSGFSLTSYGVSWIRQPPGKGL |
| | EWIGVIWHDGSTSYHSTLISRVTISRDTSKNQVSLKLSSVTAADT |
| | AVYYCASDGYSGTFAYWGQGTLVTVSS |
| 160 | QIVVTQSPATLSLSPGERATLSCTASSSVSSSYLHWYQQKPGQAP |
| | RLLIYSTSILASGIPARFSGSGSGTDFTLTISSLQPEDFAVYYCH |
| | QYHRSPYTFGQGTKLEIK |
| 161 | QVQLQESGPGLVKPSETLSLTCTVSGFSLTSYGVSWIRQPPGKGL |
| | EWIGVIWHDGSTSYHPSLKSRVTISRDTSKNQVSLKLSSVTAADT |
| | AVYYCASDGYSGTFAYWGQGTLVTVSS |
| 162 | QIVVTQSPATLSLSPGERATLSCTASSSVSSSYLHWYQQKPGQAP |
| | RLLIYSTSILASGIPARFSGSGSGTDFTLTISSLQPEDFAVYYCH |
| | QYHRSPYTFGQGTKLEIK |
| 163 | QVQLQESGPGLVKPSETLSLTCTVSGFSLSSYGVSWIRQPPGKGL |
| | EWIGVIWHDGSTNYNPSLKSRVTISRDTSKNQVSLKLSSVTAADT |
| | AVYYCASDGYSGTFAYWGQGTLVTVSS |
| 164 | QIVVTQSPATLSLSPGERATLSCTASSSVSSSYLHWYQQKPGQAP |
| | RLLIYSTSILASGIPARFSGSGSGTDFTLTISSLOPEDFAVYYCH |
| | QYHRSPYTFGQGTKLEIK |
| 168 | GFSLTSY |
| 169 | WHDGS |

1. A composition for depleting mast cells, the composition comprising:
a PPSU nanoparticle leaded with midostaurin and coated with an anti-siglec 6 antibody; and
a pharmaceutically acceptable carrier.

2. The composition of claim 1, wherein the anti-siglec 6 antibody is selected from:
antibody clone 767329; and
an antibody comprising:
a CDRH1 comprising a sequence selected from SEQ ID NOs: 89, 17, 83, 5, 11, 77, 71, 168, 53, 29, 23, 35, 41, 47, 59, 65, 135, 141, 147;
a CDRH2 comprising a sequence selected from SEQ ID NOs: 90, 18, 84, 6, 12, 78, 72, 169, 54, 30, 24, 36, 42, 48, 60, 66, 136, 142, 148;
a CDRH3 comprising a sequence selected from SEQ ID NOs: 91, 19, 85, 7, 13, 79, 73, 7, 55, 31, 25, 37, 43, 49, 61, 67, 137, 143, 149;
a CDRL1 comprising a sequence selected from SEQ ID NOs: 92, 20, 86, 8, 14, 80, 74, 8, 56, 32, 26, 38, 44, 50, 62, 68, 138, 144, 150;
a CDRL2 comprising a sequence selected from SEQ ID NOs: 93, 21, 87, 9, 15, 81, 75, 9, 57, 33, 27, 39, 45, 51, 63, 69, 139, 145, 151; and
a CDRL3 comprising a sequence selected from SEQ ID NOs: 94, 22, 88, 10, 16, 82, 76, 10, 58, 34, 28, 40, 46, 52, 64, 70, 140, 146, 152.

3. The composition of claim 2, wherein the anti-siglec 6 antibody comprises:
(a) the CDRH1 sequence comprising SEQ ID NO: 89; the CDRH2 sequence comprising SEQ ID NO: 90; the CDRH3 comprising SEQ ID NO: 91; the CDRL1 sequence comprising SEQ ID NO: 92; the CDRL2 sequence comprising SEQ ID NO: 93; and the CDRL3 sequence comprising SEQ ID NO: 94;
(b) the CDRH1 sequence comprising SEQ ID NO: 17; the CDRH2 sequence comprising SEQ ID NO: 18; the CDRH3 comprising SEQ ID NO: 19; the CDRL1 sequence comprising SEQ ID NO: 20; the CDRL2 sequence comprising SEQ ID NO: 21; and the CDRL3 sequence comprising SEQ ID NO: 22;
(c) the CDRH1 sequence comprising SEQ ID NO: 83; the CDRH2 sequence comprising SEQ ID NO: 84; the CDRH3 comprising SEQ ID NO: 85; the CDRL1 sequence comprising SEQ ID NO: 86; the CDRL2 sequence comprising SEQ ID NO: 87; and the CDRL3 sequence comprising SEQ ID NO: 88;
(d) the CDRH1 sequence comprising SEQ ID NO: 5; the CDRH2 sequence comprising SEQ ID NO: 6; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10;
(c) the CDRH1 sequence comprising SEQ ID NO: 11; the CDRH2 sequence comprising SEQ ID NO: 12; the CDRH3 comprising SEQ ID NO: 13; the CDRL1 sequence comprising SEQ ID NO: 14; the CDRL2 sequence comprising SEQ ID NO: 15; and the CDRL3 sequence comprising SEQ ID NO: 16;
(f) the CDRH1 sequence comprising SEQ ID NO: 77; the CDRH2 sequence comprising SEQ ID NO: 78; the CDRH3 comprising SEQ ID NO: 79; the CDRL1 sequence comprising SEQ ID NO: 80; the CDRL2 sequence comprising SEQ ID NO: 81; and the CDRL3 sequence comprising SEQ ID NO: 82;
(g) the CDRH1 sequence comprising SEQ ID NO: 71; the CDRH2 sequence comprising SEQ ID NO: 72; the CDRH3 comprising SEQ ID NO: 73; the CDRL1 sequence comprising SEQ ID NO: 74; the CDRL2 sequence comprising SEQ ID NO: 75; and the CDRL3 sequence comprising SEQ ID NO: 76;
(h) the CDRH1 sequence comprising SEQ ID NO: 168; the CDRH2 sequence comprising SEQ ID NO: 169; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10;
(i) the CDRH1 sequence comprising SEQ ID NO: 53; the CDRH2 sequence comprising SEQ ID NO: 54; the CDRH3 comprising SEQ ID NO: 55; the CDRL1 sequence comprising SEQ ID NO: 56; the CDRL2 sequence comprising SEQ ID NO: 57; and the CDRL3 sequence comprising SEQ ID NO: 58;
(j) the CDRH1 sequence comprising SEQ ID NO: 29; the CDRH2 sequence comprising SEQ ID NO: 30; the CDRH3 comprising SEQ ID NO: 31; the CDRL1 sequence comprising SEQ ID NO: 32; the CDRL2 sequence comprising SEQ ID NO: 33; and the CDRL3 sequence comprising SEQ ID NO: 34;
(k) the CDRH1 sequence comprising SEQ ID NO: 23; the CDRH2 sequence comprising SEQ ID NO: 24; the CDRH3 comprising SEQ ID NO: 25; the CDRL1 sequence comprising SEQ ID NO: 26; the CDRL2 sequence comprising SEQ ID NO: 27; and the CDRL3 sequence comprising SEQ ID NO: 28;
(l) the CDRH1 sequence comprising SEQ ID NO: 35; the CDRH2 sequence comprising SEQ ID NO: 36; the CDRH3 comprising SEQ ID NO: 37; the CDRL1 sequence comprising SEQ ID NO: 38; the CDRL2 sequence comprising SEQ ID NO: 39; and the CDRL3 sequence comprising SEQ ID NO: 40;
(m) the CDRH1 sequence comprising SEQ ID NO: 41; the CDRH2 sequence comprising SEQ ID NO: 42; the CDRH3 comprising SEQ ID NO: 43; the CDRL1 sequence comprising SEQ ID NO: 44; the CDRL2 sequence comprising SEQ ID NO: 45; and the CDRL3 sequence comprising SEQ ID NO: 46;
(n) the CDRH1 sequence comprising SEQ ID NO: 47; the CDRH2 sequence comprising SEQ ID NO: 48; the CDRH3 comprising SEQ ID NO: 49; the CDRL1 sequence comprising SEQ ID NO: 50; the CDRL2 sequence comprising SEQ ID NO: 51; and the CDRL3 sequence comprising SEQ ID NO: 52;
(o) the CDRH1 sequence comprising SEQ ID NO: 59; the CDRH2 sequence comprising SEQ ID NO: 60; the CDRH3 comprising SEQ ID NO: 61; the CDRL1 sequence comprising SEQ ID NO: 62; the CDRL2 sequence comprising SEQ ID NO: 63; and the CDRL3 sequence comprising SEQ ID NO: 64;
(p) the CDRH1 sequence comprising SEQ ID NO: 65; the CDRH2 sequence comprising SEQ ID NO: 66; the CDRH3 comprising SEQ ID NO: 67; the CDRL1 sequence comprising SEQ ID NO: 68; the CDRL2 sequence comprising SEQ ID NO: 69; and the CDRL3 sequence comprising SEQ ID NO: 70;
(q) the CDRH1 sequence comprising SEQ ID NO: 159; the CDRH2 sequence comprising SEQ ID NO: 160; the CDRH3 comprising SEQ ID NO: 161; the CDRL1 sequence comprising SEQ ID NO: 162; the CDRL2 sequence comprising SEQ ID NO: 163; and
the CDRL3 sequence comprising SEQ ID NO: 164;
(r) the CDRH1 sequence comprising SEQ ID NO: 135; the CDRH2 sequence comprising SEQ ID NO: 136; the CDRH3 comprising SEQ ID NO: 137; the CDRL1 sequence comprising SEQ ID NO: 138; the CDRL2 sequence comprising SEQ ID NO: 139; and
the CDRL3 sequence comprising SEQ ID NO: 140;
(s) the CDRH1 sequence comprising SEQ ID NO: 141; the CDRH2 sequence comprising SEQ ID NO: 142; the CDRH3 comprising SEQ ID NO: 143; the CDRL1 sequence comprising SEQ ID NO: 144; the CDRL2 sequence comprising SEQ ID NO: 145; and
the CDRL3 sequence comprising SEQ ID NO: 146; or
(t) the CDRH1 sequence comprising SEQ ID NO: 147; the CDRH2 sequence comprising SEQ ID NO: 148; the CDRH3 comprising SEQ ID NO: 149; the CDRL1 sequence comprising SEQ ID NO: 150; the CDRL2 sequence comprising SEQ ID NO: 151; and
the CDRL3 sequence comprising SEQ ID NO: 152.

4. A method for depleting mast cells in a subject in need thereof, the method comprising administering to the subject the composition of claim 1.

5. The method of claim 4, wherein the subject has mastocytosis, cutaneous mastocytosis, indolent systemic mastocytosis, systemic smoldering mastocytosis, aggressive systemic mastocytosis, mast cell leukemia, mast cell sarcoma, or systemic mastocytosis.

6. A method for preparing a therapeutic nanoparticle, the method comprising:
synthesizing the nanoparticle with a therapeutic compound by:
mixing a poly(propylene sulfone) (PPSU) homopolymer and the therapeutic compound in DMSO;
adding water to the DMSO; and
removing the DMSO by dialysis; and
adsorbing a targeting protein onto the surface of the nanoparticle by:
incubating the nanoparticle with the targeting protein in a buffer.

7. The method of claim 6, wherein the therapeutic compound is midostaurin.

8. The method of claim 6, wherein the targeting protein is an antibody.

9. The method of claim 8, wherein the targeting protein is an anti-siglec 6 antibody.

10. The method of claim 9, wherein the antibody is selected from:
anti-siglec-6 antibody clone 767329; and
an antibody comprising:
a CDRH1 comprising a sequence selected from SEQ ID NOs: 89, 17, 83, 5, 11, 77, 71, 168, 53, 29, 23, 35, 41, 47, 59, 65, 135, 141, 147;
a CDRH2 comprising a sequence selected from SEQ ID NOs: 90, 18, 84, 6, 12, 78, 72, 169, 54, 30, 24, 36, 42, 48, 60, 66, 136, 142, 148;
a CDRH3 comprising a sequence selected from SEQ ID NOs: 91, 19, 85, 7, 13, 79, 73, 7, 55, 31, 25, 37, 43, 49, 61, 67, 137, 143, 149;
a CDRL1 comprising a sequence selected from SEQ ID NOs: 92, 20, 86, 8, 14, 80, 74, 8, 56, 32, 26, 38, 44, 50, 62, 68, 138, 144, 150;
a CDRL2 comprising a sequence selected from SEQ ID NOs: 93, 21, 87, 9, 15, 81, 75, 9, 57, 33, 27, 39, 45, 51, 63, 69, 139, 145, 151; and
a CDRL3 comprising a sequence selected from SEQ ID NOs: 94, 22, 88, 10, 16, 82, 76, 10, 58, 34, 28, 40, 46, 52, 64, 70, 140, 146, 152.

11. The composition of claim 10, wherein the anti-siglec 6 antibody comprises:
(a) the CDRH1 sequence comprising SEQ ID NO: 89; the CDRH2 sequence comprising SEQ ID NO: 90; the CDRH3 comprising SEQ ID NO: 91; the CDRL1 sequence comprising SEQ ID NO: 92; the CDRL2 sequence comprising SEQ ID NO: 93; and the CDRL3 sequence comprising SEQ ID NO: 94;
(b) the CDRH1 sequence comprising SEQ ID NO: 17; the CDRH2 sequence comprising SEQ ID NO: 18; the CDRH3 comprising SEQ ID NO: 19; the CDRL1 sequence comprising SEQ ID NO: 20; the CDRL2 sequence comprising SEQ ID NO: 21; and the CDRL3 sequence comprising SEQ ID NO: 22;
(c) the CDRH1 sequence comprising SEQ ID NO: 83; the CDRH2 sequence comprising SEQ ID NO: 84; the CDRH3 comprising SEQ ID NO: 85; the CDRL1 sequence comprising SEQ ID NO: 86; the CDRL2 sequence comprising SEQ ID NO: 87; and the CDRL3 sequence comprising SEQ ID NO: 88;
(d) the CDRH1 sequence comprising SEQ ID NO: 5; the CDRH2 sequence comprising SEQ ID NO: 6; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10;
(e) the CDRH1 sequence comprising SEQ ID NO: 11; the CDRH2 sequence comprising SEQ ID NO: 12; the CDRH3 comprising SEQ ID NO: 13; the CDRL1 sequence comprising SEQ ID NO: 14; the CDRL2 sequence comprising SEQ ID NO: 15; and the CDRL3 sequence comprising SEQ ID NO: 16;
(f) the CDRH1 sequence comprising SEQ ID NO: 77; the CDRH2 sequence comprising SEQ ID NO: 78; the CDRH3 comprising SEQ ID NO: 79; the CDRL1 sequence comprising SEQ ID NO: 80; the CDRL2 sequence comprising SEQ ID NO: 81; and the CDRL3 sequence comprising SEQ ID NO: 82;
(g) the CDRH1 sequence comprising SEQ ID NO: 71; the CDRH2 sequence comprising SEQ ID NO: 72; the CDRH3 comprising SEQ ID NO: 73; the CDRL1 sequence comprising SEQ ID NO: 74; the CDRL2 sequence comprising SEQ ID NO: 75; and the CDRL3 sequence comprising SEQ ID NO: 76;
(h) the CDRH1 sequence comprising SEQ ID NO: 168; the CDRH2 sequence comprising SEQ ID NO: 169; the CDRH3 comprising SEQ ID NO: 7; the CDRL1 sequence comprising SEQ ID NO: 8; the CDRL2 sequence comprising SEQ ID NO: 9; and the CDRL3 sequence comprising SEQ ID NO: 10;
(i) the CDRH1 sequence comprising SEQ ID NO: 53; the CDRH2 sequence comprising SEQ ID NO: 54; the CDRH3 comprising SEQ ID NO: 55; the CDRL1 sequence comprising SEQ ID NO: 56; the CDRL2 sequence comprising SEQ ID NO: 57; and the CDRL3 sequence comprising SEQ ID NO: 58;
(j) the CDRH1 sequence comprising SEQ ID NO: 29; the CDRH2 sequence comprising SEQ ID NO: 30; the CDRH3 comprising SEQ ID NO: 31; the CDRL1 sequence comprising SEQ ID NO: 32; the CDRL2 sequence comprising SEQ ID NO: 33; and the CDRL3 sequence comprising SEQ ID NO: 34;
(k) the CDRH1 sequence comprising SEQ ID NO: 23; the CDRH2 sequence comprising SEQ ID NO: 24; the CDRH3 comprising SEQ ID NO: 25; the CDRL1 sequence comprising SEQ ID NO: 26; the CDRL2 sequence comprising SEQ ID NO: 27; and the CDRL3 sequence comprising SEQ ID NO: 28;
(l) the CDRH1 sequence comprising SEQ ID NO: 35; the CDRH2 sequence comprising SEQ ID NO: 36; the CDRH3 comprising SEQ ID NO: 37; the CDRL1 sequence comprising SEQ ID NO: 38; the CDRL2 sequence comprising SEQ ID NO: 39; and the CDRL3 sequence comprising SEQ ID NO: 40;
(m) the CDRH1 sequence comprising SEQ ID NO: 41; the CDRH2 sequence comprising SEQ ID NO: 42; the CDRH3 comprising SEQ ID NO: 43; the CDRL1 sequence comprising SEQ ID NO: 44; the CDRL2 sequence comprising SEQ ID NO: 45; and the CDRL3 sequence comprising SEQ ID NO: 46;
(n) the CDRH1 sequence comprising SEQ ID NO: 47; the CDRH2 sequence comprising SEQ ID NO: 48; the CDRH3 comprising SEQ ID NO: 49; the CDRL1 sequence comprising SEQ ID NO: 50; the CDRL2 sequence comprising SEQ ID NO: 51; and the CDRL3 sequence comprising SEQ ID NO: 52;
(o) the CDRH1 sequence comprising SEQ ID NO: 59; the CDRH2 sequence comprising SEQ ID NO: 60; the CDRH3 comprising SEQ ID NO: 61; the CDRL1 sequence comprising SEQ ID NO: 62; the CDRL2 sequence comprising SEQ ID NO: 63; and the CDRL3 sequence comprising SEQ ID NO: 64;
(p) the CDRH1 sequence comprising SEQ ID NO: 65; the CDRH2 sequence comprising SEQ ID NO: 66; the CDRH3 comprising SEQ ID NO: 67; the CDRL1 sequence comprising SEQ ID NO: 68; the CDRL2 sequence comprising SEQ ID NO: 69; and the CDRL3 sequence comprising SEQ ID NO: 70;
(q) the CDRH1 sequence comprising SEQ ID NO: 159; the CDRH2 sequence comprising SEQ ID NO: 160; the CDRH3 comprising SEQ ID NO: 161; the CDRL1 sequence comprising SEQ ID NO: 162; the CDRL2 sequence comprising SEQ ID NO: 163; and
the CDRL3 sequence comprising SEQ ID NO: 164;
(r) the CDRH1 sequence comprising SEQ ID NO: 135; the CDRH2 sequence comprising SEQ ID NO: 136; the CDRH3 comprising SEQ ID NO: 137; the CDRL1 sequence comprising SEQ ID NO: 138; the CDRL2 sequence comprising SEQ ID NO: 139; and
the CDRL3 sequence comprising SEQ ID NO: 140;
(s) the CDRH1 sequence comprising SEQ ID NO: 141; the CDRH2 sequence comprising SEQ ID NO: 142; the CDRH3 comprising SEQ ID NO: 143; the CDRL1 sequence comprising SEQ ID NO: 144; the CDRL2 sequence comprising SEQ ID NO: 145; and
the CDRL3 sequence comprising SEQ ID NO: 146; or
(t) the CDRH1 sequence comprising SEQ ID NO: 147; the CDRH2 sequence comprising SEQ ID NO: 148; the CDRH3 comprising SEQ ID NO: 149; the CDRL1 sequence comprising SEQ ID NO: 150; the CDRL2 sequence comprising SEQ ID NO: 151; and
the CDRL3 sequence comprising SEQ ID NO: 152.

12. The method of claim 6, wherein the step of adding water to the DMSO comprises adding the water at a ratio of about 2:1 to about 10:1.

13. The method of claim 12, wherein the water is added in at least two increments, wherein each increment comprises a ratio of about 2:1 to the DMSO.

14. The method of claim 6, wherein the therapeutic compound is at between about 1.2 wt % and about 5 wt %.

15. The method of claim 14, wherein the therapeutic compound is at about 2.5 wt %.

16. The method of claim 6, wherein the step of incubating the nanoparticle with the targeting protein in the buffer is done for between about 1 second and about 5 minutes at between about 20° C. and about 22° C.

17. The method of claim 6, wherein the targeting protein is incubated at a higher concentration than the nanoparticle.

18. The method of claim 6, wherein the targeting protein is incubated at between about 0.001 wt % and about 1000 wt % PPSU.

19. The method of claim 6, wherein the PPSU homopolymer comprises PPSU₂₀.

20. A therapeutic nanoparticle prepared by the method of claim 6.


SEQUENCE LISTING (169 sequences)

SEQ ID NO: 1 - protein, 453 aa, Homo sapiens
MQGAQEASASEMLPLLLPLLWAGALAQERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGVLGAVWGASITTLVFLCVCFIFRVKTRRKKAAQPVQNTDDVNPVMVSGSRGHQHQFQTGIVSDHPAEAGPISEDEQELHYAVLHFHKVQPQEPKVTDTEYSEIKIHK

SEQ ID NO: 2-4 - skipped (3 entries)

SEQ ID NO: 5 - protein, 10 aa, synthetic construct
GFSLTSYGVS

SEQ ID NO: 6 - protein, 16 aa, synthetic construct
VIWHDGSTSYHSTLIS

SEQ ID NO: 7 - protein, 9 aa, synthetic construct
DGYSGTFAY

SEQ ID NO: 8 - protein, 12 aa, synthetic construct
TASSSVSSSYLH

SEQ ID NO: 9 - protein, 7 aa, synthetic construct
STSILAS

SEQ ID NO: 10 - protein, 9 aa, synthetic construct
HQYHRSPYT

SEQ ID NO: 11 - protein, 10 aa, synthetic construct
GYIFTSYWMY

SEQ ID NO: 12 - protein, 17 aa, synthetic construct
NVNPYSGDTNYNEKFKT

SEQ ID NO: 13 - protein, 11 aa, synthetic construct
GDGLYYYAMDY

SEQ ID NO: 14 - protein, 11 aa, synthetic construct
KASQDINSYLT

SEQ ID NO: 15 - protein, 7 aa, synthetic construct
RASRLVD

SEQ ID NO: 16 - protein, 9 aa, synthetic construct
LQYDEFPLT

SEQ ID NO: 17 - protein, 10 aa, synthetic construct
GYTFTNYYIH

SEQ ID NO: 18 - protein, 17 aa, synthetic construct
MIHPISGSTNYNEKLKI

SEQ ID NO: 19 - protein, 12 aa, synthetic construct
RYYGSSYWYFDV

SEQ ID NO: 20 - protein, 10 aa, synthetic construct
RASSSVSYMN

SEQ ID NO: 21 - protein, 7 aa, synthetic construct
ATSNLAS

SEQ ID NO: 22 - protein, 9 aa, synthetic construct
QQWSSHPPT

SEQ ID NO: 23 - protein, 10 aa, synthetic construct
GYTFTTYGMS

SEQ ID NO: 24 - protein, 17 aa, synthetic construct
WINTYSGVPTYADDFKG

SEQ ID NO: 25 - protein, 10 aa, synthetic construct
LAYFGYYFDF

SEQ ID NO: 26 - protein, 11 aa, synthetic construct
KASQNVGSAVA

SEQ ID NO: 27 - protein, 7 aa, synthetic construct
SASNRYT

SEQ ID NO: 28 - protein, 9 aa, synthetic construct
QQYSSYPYT

SEQ ID NO: 29 - protein, 10 aa, synthetic construct
GFSFSTYVMS

SEQ ID NO: 30 - protein, 17 aa, synthetic construct
SISGGGSNTYYPDSVKG

SEQ ID NO: 31 - protein, 11 aa, synthetic construct
REVVVYWYFDV

SEQ ID NO: 32 - protein, 11 aa, synthetic construct
RASENIYSNLA

SEQ ID NO: 33 - protein, 7 aa, synthetic construct
SAKNLAD

SEQ ID NO: 34 - protein, 9 aa, synthetic construct
QHFWGTPLT

SEQ ID NO: 35 - protein, 10 aa, synthetic construct
GFSLTSYGVS

SEQ ID NO: 36 - protein, 16 aa, synthetic construct
IIWGDGSTNYHSALIS

SEQ ID NO: 37 - protein, 9 aa, synthetic construct
GLSDWYFDV

SEQ ID NO: 38 - protein, 10 aa, synthetic construct
RASSSVSYMH

SEQ ID NO: 39 - protein, 7 aa, synthetic construct
ATSNLAS

SEQ ID NO: 40 - protein, 9 aa, synthetic construct
QQWSSNPYT

SEQ ID NO: 41 - protein, 10 aa, synthetic construct
GYTFTTYGMS

SEQ ID NO: 42 - protein, 17 aa, synthetic construct
WINTYSGVPTYADDFKG

SEQ ID NO: 43 - protein, 10 aa, synthetic construct
LAYFGYYFDF

SEQ ID NO: 44 - protein, 11 aa, synthetic construct
KASQNVGSAVT

SEQ ID NO: 45 - protein, 7 aa, synthetic construct
SASNRYT

SEQ ID NO: 46 - protein, 9 aa, synthetic construct
QQYSSYPYT

SEQ ID NO: 47 - protein, 10 aa, synthetic construct
GFSLTSYGVH

SEQ ID NO: 48 - protein, 16 aa, synthetic construct
VIWGGGSTDYNAGFIS

SEQ ID NO: 49 - protein, 9 aa, synthetic construct
ALSDWYFDV

SEQ ID NO: 50 - protein, 10 aa, synthetic construct
RASSSVSYMH

SEQ ID NO: 51 - protein, 7 aa, synthetic construct
ATSNLAS

SEQ ID NO: 52 - protein, 9 aa, synthetic construct
QQWSSNPLT

SEQ ID NO: 53 - protein, 10 aa, synthetic construct
GYTFTDYYMN

SEQ ID NO: 54 - protein, 17 aa, synthetic construct
DINPKIGDTIYNQKFKG

SEQ ID NO: 55 - protein, 11 aa, synthetic construct
WDYYGSSLFDY

SEQ ID NO: 56 - protein, 11 aa, synthetic construct
KASQDVTTAVA

SEQ ID NO: 57 - protein, 7 aa, synthetic construct
WASTRHT

SEQ ID NO: 58 - protein, 9 aa, synthetic construct
QQHYSTPYT

SEQ ID NO: 59 - protein, 10 aa, synthetic construct
GYTLTTYGMS

SEQ ID NO: 60 - protein, 17 aa, synthetic construct
WINTYSGVPTYADEFKG

SEQ ID NO: 61 - protein, 10 aa, synthetic construct
LGYYGYYFDY

SEQ ID NO: 62 - protein, 11 aa, synthetic construct
RASQSIRNNLH

SEQ ID NO: 63 - protein, 7 aa, synthetic construct
YIFQSIS

SEQ ID NO: 64 - protein, 10 aa, synthetic construct
QQSDSWPHVT

SEQ ID NO: 65 - protein, 10 aa, synthetic construct
GFSLTSYGVN

SEQ ID NO: 66 - protein, 16 aa, synthetic construct
VIWGDGSTNYHSALIS

SEQ ID NO: 67 - protein, 9 aa, synthetic construct
ALSDWYFDV

SEQ ID NO: 68 - protein, 10 aa, synthetic construct
RASSSVSYMH

SEQ ID NO: 69 - protein, 7 aa, synthetic construct
ATSNLAS

SEQ ID NO: 70 - protein, 9 aa, synthetic construct
QQWSSNPYT

SEQ ID NO: 71 - protein, 10 aa, synthetic construct
GYTFTDYKMH

SEQ ID NO: 72 - protein, 17 aa, synthetic construct
NINPNNGGTSYNQKFKG

SEQ ID NO: 73 - protein, 12 aa, synthetic construct
RYYGSSSSYFDY

SEQ ID NO: 74 - protein, 10 aa, synthetic construct
SVSSSVSYMH

SEQ ID NO: 75 - protein, 7 aa, synthetic construct
DTSKLAS

SEQ ID NO: 76 - protein, 9 aa, synthetic construct
QQWSSNPPT

SEQ ID NO: 77 - protein, 10 aa, synthetic construct
GFTFSDYGIH

SEQ ID NO: 78 - protein, 17 aa, synthetic construct
YITSVSSTIYYADTVKG

SEQ ID NO: 79 - protein, 7 aa, synthetic construct
LDWYFDV

SEQ ID NO: 80 - protein, 10 aa, synthetic construct
SANSSVSFMH

SEQ ID NO: 81 - protein, 7 aa, synthetic construct
DTSKLAS

SEQ ID NO: 82 - protein, 9 aa, synthetic construct
QQWSSNPLT

SEQ ID NO: 83 - protein, 10 aa, synthetic construct
GYTFTSYWMH

SEQ ID NO: 84 - protein, 17 aa, synthetic construct
TIYPGNSDSTYNQKFKG

SEQ ID NO: 85 - protein, 13 aa, synthetic construct
EGTVVANYYGLDY

SEQ ID NO: 86 - protein, 10 aa, synthetic construct
RASSSVSYIH

SEQ ID NO: 87 - protein, 7 aa, synthetic construct
ATSNLAS

SEQ ID NO: 88 - protein, 9 aa, synthetic construct
QQWSSKPPT

SEQ ID NO: 89 - protein, 10 aa, synthetic construct
GFSLTSYSVH

SEQ ID NO: 90 - protein, 16 aa, synthetic construct
VIWSGGSTDYNAVFIS

SEQ ID NO: 91 - protein, 8 aa, synthetic construct
YSYYTMDY

SEQ ID NO: 92 - protein, 16 aa, synthetic construct
RSSQSLVLDNGNTYLH

SEQ ID NO: 93 - protein, 7 aa, synthetic construct
KVSNRFS

SEQ ID NO: 94 - protein, 9 aa, synthetic construct
SQSTHVPFT

SEQ ID NO: 95-134 - skipped (40 entries)

SEQ ID NO: 135 - protein, 10 aa, synthetic construct
GFNIKDDYMH

SEQ ID NO: 136 - protein, 17 aa, synthetic construct
WIDLENGDTEYASKFQG

SEQ ID NO: 137 - protein, 12 aa, synthetic construct
PIGTVVAEGFAY

SEQ ID NO: 138 - protein, 11 aa, synthetic construct
RASENIYSNLA

SEQ ID NO: 139 - protein, 7 aa, synthetic construct
AATNLAD

SEQ ID NO: 140 - protein, 9 aa, synthetic construct
QHFWGTPYT

SEQ ID NO: 141 - protein, 10 aa, synthetic construct
GYTFTDYKMH

SEQ ID NO: 142 - protein, 17 aa, synthetic construct
NINPNNGGTSYNQKFKG

SEQ ID NO: 143 - protein, 12 aa, synthetic construct
RYYGSSSSYFDY

SEQ ID NO: 144 - protein, 11 aa, synthetic construct
RASQSIGTSIH

SEQ ID NO: 145 - protein, 7 aa, synthetic construct
YASESIS

SEQ ID NO: 146 - protein, 9 aa, synthetic construct
QQSNSWPTT

SEQ ID NO: 147 - protein, 10 aa, synthetic construct
GFTFSDYGMH

SEQ ID NO: 148 - protein, 17 aa, synthetic construct
YISSDSSTIYYADTVKG

SEQ ID NO: 149 - protein, 10 aa, synthetic construct
TIYYGNYFDY

SEQ ID NO: 150 - protein, 12 aa, synthetic construct
SASSSVSSSSLY

SEQ ID NO: 151 - protein, 7 aa, synthetic construct
SMSNLAS

SEQ ID NO: 152 - protein, 9 aa, synthetic construct
QQWSSNPLT

SEQ ID NO: 153-158 - skipped (6 entries)

SEQ ID NO: 159 - protein, 117 aa, synthetic construct
QVQLQESGPGLVKPSETLSLTCTVSGFSLTSYGVSWIRQPPGKGLEWIGVIWHDGSTSYHSTLISRVTISRDTSKNQVSLKLSSVTAADTAVYYCASDGYSGTFAYWGQGTLVTVSS

SEQ ID NO: 160 - protein, 108 aa, synthetic construct
QIVVTQSPATLSLSPGERATLSCTASSSVSSSYLHWYQQKPGQAPRLLIYSTSILASGIPARFSGSGSGTDFTLTISSLQPEDFAVYYCHQYHRSPYTFGQGTKLEIK

SEQ ID NO: 161 - protein, 117 aa, synthetic construct
QVQLQESGPGLVKPSETLSLTCTVSGFSLTSYGVSWIRQPPGKGLEWIGVIWHDGSTSYHPSLKSRVTISRDTSKNQVSLKLSSVTAADTAVYYCASDGYSGTFAYWGQGTLVTVSS

SEQ ID NO: 162 - protein, 108 aa, synthetic construct; identical to SEQ ID NO: 160

SEQ ID NO: 163 - protein, 117 aa, synthetic construct
QVQLQESGPGLVKPSETLSLTCTVSGFSLSSYGVSWIRQPPGKGLEWIGVIWHDGSTNYNPSLKSRVTISRDTSKNQVSLKLSSVTAADTAVYYCASDGYSGTFAYWGQGTLVTVSS

SEQ ID NO: 164 - protein, 108 aa, synthetic construct; identical to SEQ ID NO: 160

SEQ ID NO: 165-167 - skipped (3 entries)

SEQ ID NO: 168 - protein, 7 aa, synthetic construct
GFSLTSY

SEQ ID NO: 169 - protein, 5 aa, synthetic construct
WHDGS